(19) United States
(12) Patent Application Publication
(10) Pub. No.: US 2024/0099335 A1
(43) Pub. Date: Mar. 28, 2024

(54) METHODS OF SELECTIVELY PROMOTING ANIMAL WELFARE THROUGH MODULATION OF MICROBIOME

(71) Applicants: DSM IP Assets B.V., Heerlen (NL); NOVOZYMES A/S, Bagsvaerd (DK)

(72) Inventors: Joshua CLAYPOOL, Kaiseraugst (CH); Aaron COWIESON, Kaiseraugst (CH); Kevin FREEMAN, Kaiseraugst (CH); Ghislain SCHYNS, Kaiseraugst (CH)

(21) Appl. No.: 18/263,809

(22) PCT Filed: Feb. 15, 2022
(86) PCT No.: PCT/EP2022/053674

§ 371 Date: Aug. 1, 2023

Related U.S. Application Data
(60) Provisional application No. 63/149,808, filed on Feb. 16, 2021.

(51) Int. Cl. A23K 20/189; A23K 50/75

(52) U.S. Cl. CPC A23K 20/189; A23K 50/75; C12Y 302/01092

(57) ABSTRACT

The present disclosure relates to methods of feeding animals by providing feed additives that modulate the gut micro-biome improve the health and welfare of the animal. The present disclosure further relates to methods of modulating level of secondary metabolites present in the gastrointestinal tract of an animal. Such modulation includes, for example, modulating the level of tryptophan metabolism derivates.

### TECHNICAL FIELD

The present invention pertains to a method for improving the health of production animals. In particular the invention pertains to methods for improving the welfare of production animals, decreasing systemic inflammation of production animals, decreasing local inflammation of production animals, and reducing the light regimen into the daily circadian rhythm. The improvement of the health of production animals is achieved by feeding the animals with food which can regulate the tryptophan-derived metabolites in the gut or blood of the animal.

### BACKGROUND INFORMATION

Raising of production animals (livestock) has been largely industrialized. Animals are raised in large flocks within a confined space. Feeding of the animals is highly adjusted to maximize the growth of meat of the animal as is light and climate control. With the help of science and modern technology, it was made possible to shorten the time period of raising production animals and at the same time maximize the meat production. However, such hastened growth does generate many problems to the animal. It has been observed that raising a large flock of animals in a confined space, if done improperly, could harm the social welfare of the animal. For example, animals such as chickens may develop social disturbance behavior such as feather pecking against their peers. In another example, chickens which have been subjected to prolonged illumination time have social disturbance behaviors. It was known that illumination is an important factor affecting the circadian rhythms of animals. Long time exposure to the light cycle can change the circadian rhythm systems of animals and thus affect the health of their reproduction, metabolism, immunity and nerve systems (Wang et al., 2002, PeerJ, DOI 10.7717/peerj.9638). Thus, there is a need for a method of improving the health of production animals which are raised in a confined space and an accelerated growth schedule. There is further a need to solve this problem by not using complicated and inorganic solutions such as medicine or genetic engineering, but a much simpler and low-cost solution.

Secondary metabolism refers to pathways and small molecule products of metabolism that are involved in ecological interactions. Unlike primary metabolism which is absolutely required for the survival of the organism, secondary metabolisms play a major role in the adaptation of organisms to their environment. Secondary metabolism occurs mainly in bacteria during the stationary phase of growth and is concomitant with a switch in energy and carbon flux away from biomass production toward the production of small, bioactive molecules (secondary metabolites) (Ruiz et al., 2010, Critical Reviews in Microbiology, Vol 36, Issue 2, pp146-167,). In the context of the production animals, the secondary metabolites produced by the microbiome residing in the digestive system of its host animal are very important for interspecies communication and behavior of both the microbiome and its host.

Traditionally, the approaches for improving the health of production animals were focusing on direct intervention with the organs of the animal by means of pharmaceuticals. Given the increasing knowledge about secondary metabolism and metabolites, there is a need to identify novel ways of enhancing the health of production animals by influencing the microbiome in the gut of the animal. In other words, there is a need to identify novel ways of influencing the production of secondary metabolites which are produced by microbiome and able to regulate the behavior of the host animal.

### SUMMARY OF THE INVENTION

The present invention is directed to a method for improving the health of a group of production animals kept in a confined space, the method comprising increasing the ratio of kynurenine:tryptophan in the body of said group of animals by feeding said group of production animals one of more of the following feed additives: N-acetyl-muramidase, and protease, wherein the ratio of kynurenine:tryptophan in the digestive system of said group of animals is increased for at least 10% higher than the ratio of kynurenine:tryptophan in the body of a control group of animals which are fed with the same diet except for said feed additives. In a preferred embodiment, the N-acetyl-muramidase is selected from the group consisting of: (a) a polypeptide having at least 80% sequence identity to any one of SEQ ID NOs: 1-71; (b) a variant of a polypeptide having any one of SEQ ID NOs: 1-71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions; (c) a polypeptide comprising the polypeptide of (a) or (b) and a N-terminal and/or C-terminal extension of between 1 and 10 amino acids; and (d) a fragment of a polypeptide of (a) or (b) having muramidase activity and having at least 90% of the length of the mature polypeptide; and the protease is selected from the group consisting of: (a′) a polypeptide having a sequence identity of at least 70% to any one of SEQ ID NOs 72-76; (b′) a variant of any one of SEQ ID NOs: 72-76, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions; (c′) a polypeptide comprising the polypeptide of (a′) or (b′) and a N-terminal and/or C-terminal His-tag and/or HQ-tag; (d′) a polypeptide comprising the polypeptide of (a′) or (b′) and a N-terminal and/or C-terminal extension of up to 10 amino acids, e.g. 1, 2, 3, 4, 5, 6, 7, 8, 9 or 10 amino acids; and (e′) a fragment of the polypeptide of (a′) or (b′) having protease activity and having at least 90% of the length of the mature polypeptide. In one embodiment, the ratio of kynurenine:tryptophan is measured in the feces or blood of said animals. In some embodiments, improvement of health comprises providing one of more of the following benefits to the production animals: improving the welfare of the production animals, decreasing systemic inflammation of the production animals, decreasing local inflammation of the production animals, and restoring the light regimen to the daily circadian rhythm of the production animals. Examples of improvement of welfare include reducing social disturbance and reducing feather pecking among the production animals.

The present invention is also directed to a method for improving the health of a group of production animals kept in a confined space, the method comprising increasing the ratio of peripheral serotonin:tryptophan in the digestive system of said group of animals by feeding said group of production animals one of more of the following feed additives: N-acetyl-muramidase, and protease, wherein the ratio of peripheral serotonin:tryptophan in the brain of said group of animals is increased for at least 20% higher than the ratio of peripheral serotonin:tryptophan in the digestive system of a control group. In a preferred embodiment, the N-acetyl-muramidase is selected from the group consisting of: (a) a polypeptide having at least 80% sequence identity to any one of SEQ ID NOs: 1-71; (b) a variant of a polypeptide having any one of SEQ ID NOs: 1-71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions; (c) a polypeptide comprising the polypeptide of (a) or (b) and a N-terminal and/or C-terminal extension of between 1 and 10 amino acids; and (d) a fragment of a polypeptide of (a) or (b) having muramidase activity and having at least 90% of the length of the mature polypeptide; and the protease is selected from the group consisting of: (a′) a polypeptide having a sequence identity of at least 70% to any one of SEQ ID NOs 72-76; (b′) a variant of any one of SEQ ID NOs: 72-76, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions; (c′) a polypeptide comprising the polypeptide of (a′) or (b′) and a N-terminal and/or C-terminal His-tag and/or HQ-tag; (d′) a polypeptide comprising the polypeptide of (a′) or (b′) and a N-terminal and/or C-terminal extension of up to 10 amino acids, e.g. 1, 2, 3, 4, 5, 6, 7, 8, 9 or 10 amino acids; and (e′) a fragment of the polypeptide of (a′) or (b′) having protease activity and having at least 90% of the length of the mature polypeptide.

The present invention is further directed to a method for improving the health of a group of production animals kept in a confined space, the method comprising increasing the ratio of melatonin:tryptophan in the digestive system of said group of animals by feeding said group of production animals one of more of the following group of feed additives: N-acetyl-muramidase, and protease, wherein the ratio of melatonin:tryptophan in the digestive system of said group of animals is increased for at least 10% higher than the ratio of melatonin:tryptophan in the digestive system of a control group of animals which are fed with the same diet except for said group of feed additives. In a preferred embodiment, the N-acetyl-muramidase is selected from the group consisting of: (a) a polypeptide having at least 80% sequence identity to any one of SEQ ID NOs: 1-71; (b) a variant of a polypeptide having any one of SEQ ID NOs: 1-71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions; (c) a polypeptide comprising the polypeptide of (a) or (b) and a N-terminal and/or C-terminal extension of between 1 and 10 amino acids; and (d) a fragment of a polypeptide of (a) or (b) having muramidase activity and having at least 90% of the length of the mature polypeptide; and the protease is selected from the group consisting of: (a′) a polypeptide having a sequence identity of at least 70% to any one of SEQ ID NOs 72-76; (b′) a variant of any one of SEQ ID NOs: 72-76, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions; (c′) a polypeptide comprising the polypeptide of (a′) or (b′) and a N-terminal and/or C-terminal His-tag and/or HQ-tag; (d′) a polypeptide comprising the polypeptide of (a′) or (b′) and a N-terminal and/or C-terminal extension of up to 10 amino acids, e.g. 1, 2, 3, 4, 5, 6, 7, 8, 9 or 10 amino acids; and (e′) a fragment of the polypeptide of (a′) or (b′) having protease activity and having at least 90% of the length of the mature polypeptide.

In one embodiment, the ratio of melatonin:tryptophan or serotonin:tryptophan is measured in the feces or blood of said animals. In some embodiments, improvement of health comprises providing one of more of the following benefits to the production animals: improving the welfare of the production animals, decreasing systemic inflammation of the production animals, decreasing local inflammation of the production animals, and restoring the light regimen to the daily circadian rhythm of the production animals. Examples of improvement of welfare include reducing social disturbance, reducing feather pecking among the production animals, and restoring the natural photoperiod of said group of production animals.

The present invention is also directed to a method for improving the health of a group of production animals kept in a confined space, the method comprising decreasing the ratio of tryptamine:tryptophan in the digestive system of said group of animals by feeding said group of production animals one of more of the following feed additives: N-acetyl-muramidase, and protease, wherein the ratio of tryptamine:tryptophan in the digestive system of said group of animals is decreased for at least 20% lower than the ratio of tryptamine:tryptophan in the digestive system of a control group of animals which are fed with the same diet except for said feed additives. In a preferred embodiment, the N-acetyl-muramidase is selected from the group consisting of: (a) a polypeptide having at least 80% sequence identity to any one of SEQ ID NOs: 1-71; (b) a variant of a polypeptide having any one of SEQ ID NOs: 1-71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions; (c) a polypeptide comprising the polypeptide of (a) or (b) and a N-terminal and/or C-terminal extension of between 1 and 10 amino acids; and (d) a fragment of a polypeptide of (a) or (b) having muramidase activity and having at least 90% of the length of the mature polypeptide; and the protease is selected from the group consisting of: (a′) a polypeptide having a sequence identity of at least 70% to any one of SEQ ID NOs 72-76; (b′) a variant of any one of SEQ ID NOs: 72-76, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions; (c′) a polypeptide comprising the polypeptide of (a′) or (b′) and a N-terminal and/or C-terminal His-tag and/or HQ-tag; (d′) a polypeptide comprising the polypeptide of (a′) or (b′) and a N-terminal and/or C-terminal extension of up to 10 amino acids, e.g. 1, 2, 3, 4, 5, 6, 7, 8, 9 or 10 amino acids; and (e′) a fragment of the polypeptide of (a′) or (b′) having protease activity and having at least 90% of the length of the mature polypeptide.

In one embodiment, the ratio of tryptamine:tryptophan is measured in the feces or blood of said animals. In some embodiments, improvement of health comprises providing one of more of the following benefits to the production animals: improving the welfare of the production animals, decreasing systemic inflammation of the production animals, decreasing local inflammation of the production animals, and restoring the light regimen to the daily circadian rhythm of the production animals. In some embodiments, improving performance of said group of production animals comprises providing one of more of the following benefits to said group of production animals: improving nutrient absorption, reduce gut peristaltic motility, improving vitamin absorption, and improving feed enzymatic processing. Examples of improvement of welfare include reducing social disturbance and reducing feather pecking among the production animals.

### BRIEF DESCRIPTION OF DRAWINGS

FIG. 1 is a diagram showing the pathways of tryptophan metabolism in animals. It is adopted from Liu et al., 2020, Trends in Endocrinology and Metabolsim 31: 818-833.

FIG. 2 is a graph showing the comparison of abundance of tryptophan in chicken cecum slurry when the chicken is fed with a diet supplemented with Ronozyme ProAct protease and a control diet.

FIG. 3 is a graph showing the comparison of ratio of tryptophan metabolites: tryptophan in chicken cecum slurry when the chicken is fed with a diet supplemented with Ronozyme ProAct protease and a control diet.

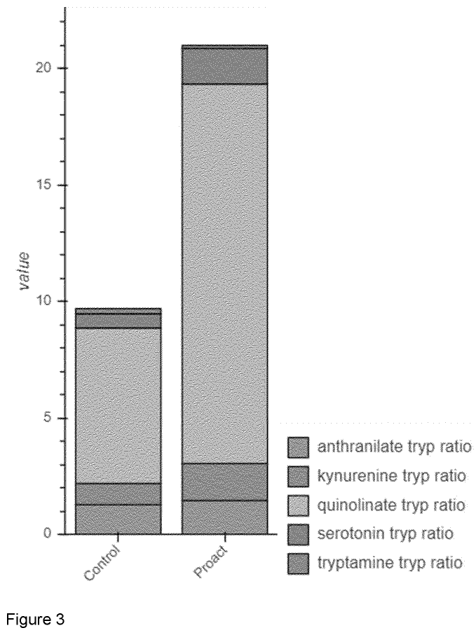

### DETAILED DESCRIPTION OF THE INVENTION

### Definitions

A production animal (also referred to as livestock) is any animal that is kept to raise meat, fiber, protein, milk, eggs, wool, skin or other products for use by humans, as opposed to companion animals which are kept for primarily for a person's company, protection, or entertainment. The keeping of production animals includes day-to-day care, selective breeding, and the raising of animals. Typical production animals are swine, bovine, fish, sheep and poultry.

A confined space can be any closed or semi-closed area designed to restrict, and preferably prevent, the free movement of an animal to an area outside of the confined space, such as a stable, paddock, fenced land, a container, sea pen etc.

Animal welfare means how an animal is coping with the conditions in which it lives. An animal is in a good state of welfare if it is healthy, comfortable, well nourished, safe, able to express innate behavior, and if it is not suffering from unpleasant states such as pain, fear, and distress. Parameters by which animal welfare can be measured are the general impression the animal provides, the presence of wounds, its ability to freely move, the number of dead animals in the neighborhood of the animal, the presence of bite marks, the presence of feather pecking behavior etc.

Raising animals means the production of animals, regardless of the purpose. Thus, “raising animals” includes raising animals for meat and/or egg production. Chickens that are bred for meat production are broiler chickens.

### Method of Improving the Health of Production Animals

In this invention, a method of improving the health of a group of production animals is shown. A preferred embodiment of the method of the invention relates to a method of improving the health of a group of production animals by modulating the amount of secondary metabolites. An also preferred embodiment of the method of the invention relates to a method of improving the health of a group of production animals by modulating the amount of one or more secondary metabolites which are produced in related metabolism pathways. In a specific embodiment, the above secondary metabolites are tryptophan derivatives. An also preferred embodiment of the method of the invention relates to a method of improving the health of a group of production animals by influencing the ratio of one of more of the following pairs of secondary metabolites: kynurenine:tryptophan, serotonin:tryptophan, melatonin:tryptophan, and tryptamine:tryptophan.

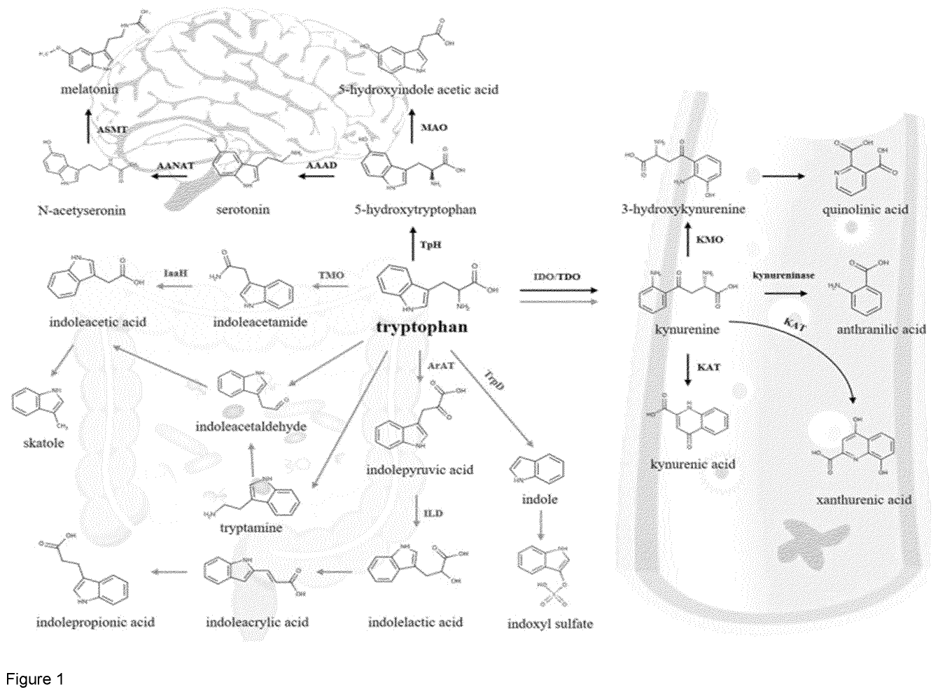

Tryptophan (Trp or Tryp) is an essential amino acid involved in the metabolic pathways for serotonin and subsequently melatonin and for nicotinamide adenine dinucleotide (NAD+). Tryptophan's fate is represented in FIG. 1. In humans, partitioning of the kynurenergic pathway and serotonergic pathway is reported to stand at 90%:10% of the tryptophan pool. Tryptophan can also produce the neuromodulator tryptamine. Tryptamine is a trace amine neuro-modulator (Gao et al. 2018 Front Cell Infect Microbiol 8:13), similar to the cathecholamine neurotransmitters. Trace amines have effects both on the central nervous system (and are therefore involved in the so-called gut-brain axis), but also in the gut lumen where they act on enterocytes. As a trace amine, tryptamine is believed to act as agonist on trace amine-associated receptor TAAR1, involved into energy metabolism and immunomodulation, thereby mediating a host-nutrition-microbiota dialog (Gainetdinov et al. 2018 Pharmacol Rev 70 (3):549-620).

Surprisingly, inventors of present application have found that a few selected nutritional interventions termed eubiotics such as N-acetyl-muramidase, and protease can cause an increased presence of certain secondary metabolites, such as tryptophan derivatives, in the gut and blood of the host animal. In other words, important catabolic metabolites of tryptophan, such as tryptamine, anthranilate, kynurenine, serotonin and melatonin have been seen in this invention to be either positively or negatively associated with nutritional interventions in a metabolomics study. The selected nutritional interventions, such as adding N-acetyl-muramidase, and protease in the feed, cause the microbiome of the host animal to modulate (increase or decrease) the amounts of secondary metabolites such as tryptophan derivatives. These derivative compounds subsequently regulate the physiological and psychological functions of the host animal and thus improve the health and welfare of the host animal.

It has been observed in the present invention that the health of the host animal is improved in four aspects. First, welfare of the group of production animals is improved. It is a common problem for monogastric animals such as chicken and ducks raised in a confined space to develop social disturbance behaviors such as feather pecking or tail biting. Disturbance behaviors like this cause poor welfare of the production animal and thus has been a persisting problem for animal farmers. The method according to the invention helps to improve the welfare of animals.

Second, the health of the host animal can be improved by way of decreasing systemic inflammation of the animal. Systemic inflammation is the result of release of pro-inflammatory cytokines from immune-related cells and the chronic activation of the innate immune system. It contributes to the development of chronical disease conditions in animals. The method according to the invention helps to reduce systemic inflammation of the animal.

Third, the health of the host animal can be improved by way of decreasing local inflammation of the animal. Local inflammation occurs within the area affected by the harmful stimulus. Acute local inflammation develops within minutes or hours following a harmful stimulus, has a short duration, and primarily involves the innate immune system. The method according to the invention helps to reduce local inflammation of the animal.

Fourth, the health of the host animal can be improved by way of reducing the light regimen/duration into the daily circadian rhythm of the animal (Soliman and Hassan 2019 Veterinary World 12(7): 1052-1059). The circadian rhythms associated with light have important effects on the growth of production animals. In the production animal farming business, one way for increasing the growth rate and meat production is by prolongation of the illumination. In some extreme cases, the illumination on poultry is extended to 23 hours a day, leaving the poultry under darkness for only one hour a day. Although such a method may increase productivity, it has negative impacts on the health as well as the welfare of the animal. It has been observed that the melatonin level of chicken under the 23 hours light and 1 hour darkness period treatment was lowered to less than half of the amount of melatonin of the chicken which are under the 16 hours light and 8 hours darkness period treatment. The method according to the present invention helps to increase the amount of melatonin and its precursor serotonin and thus restore the level of melatonin in animals which are subjected to prolonged illumination. Since artificially prolonged photoperiod leads to abnormal behavior such as aggressive interactions (tail biting, feather pecking, mobility/motility issues etc.) in poultry, restoring of melatonin level in such animals helps to improve the welfare of the animals.

It has been observed in the present invention that the health benefits described above can be achieved by increasing the ratio of kynurenine:tryptophan in the body of production animals. Kynurenine is known as a neuromodulator of stress. Birkl et al. (2020, Front Vet Sci 6:209) has monitored the kynurenine/tryptophan ratio in relation to feather pecking specifically, and more generally social disturbance in laying hens. They have found that lower KYN/TRP ratio are linked to higher social disturbance profile. It is reasoned by the inventors of the present application that an increase of kynurenine/tryptophan ratio could reduce social disturbance behavior, and this produces a positive effect on the animal welfare. Surprisingly, the inventors of the present application identified a number of selected feed additives which can increase the kynurenine/tryptophan ratio in the body of production animals, and thus improve the health and welfare of the animals.

In one embodiment of the method according to the invent, the health and welfare benefits described above can be achieved by increasing the ratio of kynurenine:tryptophan in the body of production animals at least 10% higher than the ratio of kynurenine:tryptophan in the body of a control group of animals. In some embodiments, the increase of kynurenine:tryptophan ratio is at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, or at least 40%. In some embodiment, the test group of animals is fed with a group of feed additives comprising one or more of N-acetyl-muramidase, and protease.

It has been observed in the present invention that the health and welfare benefits described above can be achieved by increasing the ratio of peripheral serotonin:tryptophan in the body of production animals. In one embodiment of the method according to the invent, the health benefits described above can be achieved by increasing the ratio of serotonin:tryptophan in the body of production animals for at least 10% higher than the ratio of serotonin:tryptophan in the body of a control group of animals. In some embodiments, the increase of serotonin:tryptophan ratio is at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, or at least 40%. In some embodiment, the test group of animals is fed with a group of feed additives comprising one or more of N-acetyl-muramidase, and protease.

Serotonin within the central nervous system cannot cross the blood/brain barrier, but tryptophan can. Therefore, higher tryptophan in the gut means more tryptophan will cross the blood/brain barrier and be transformed into central serotonin. Serotonin is the precursor of melatonin. An increase in serotonin level will cause the increase in melatonin level.

It is known that melatonin and its precursor serotonin can impact the production of insulin and glucagon. An increase in the melatonin concentration can enhance the level of insulin and glucagon in animal body. It is also known that increased levels of insulin and glucagon enhance the synthesis of fat.

Both insulin and melatonin are involved in regulating circadian rhythm (Wang et al., 2020 PeerJ 8:e9638). Change in the light cycle affect the level of insulin and melatonin produced by the animal. The changed level of insulin and melatonin in the body of the animal in turn regulates the animal's physiological response to the light cycle change. Poultry production in general, and broiler rearing process is now going to long light time, as much as 23 hours a day.

This illumination regimen strongly impacts production performance such as faster fat gain but is detrimental to animal welfare. Inventors of the present application has discovered that by compensating melatonin production through feeding animal as described herein, a stronger serotonergic flux is going into more melatonin and thus a reduction of the illumination regimen and a better animal welfare can be achieved.

It has been observed in the present invention that the health and welfare benefits described above can be achieved by increasing the ratio of melatonin:tryptophan in the body of production animals. In one embodiment of the method according to the inventors, the health benefits described above can be achieved by increasing the ratio of melatonin:tryptophan in the body of production animals for at least 10% higher than the ratio of melatonin:tryptophan in the body of a control group of animals. In some embodiments, the increase of melatonin:tryptophan ratio is at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, or at least 40%. In some embodiment, the test group of animals is fed with a group of feed additives comprising one or more of N-acetyl-muramidase, and protease.

It has been observed in the present invention that the health and welfare benefits described above can be achieved by decreasing the ratio of tryptamine:tryptophan in the body of production animals. In one embodiment of the method according to the invent, the health benefits described above can be achieved by decreasing the ratio of tryptamine:tryptophan in the body of production animals for at least 10% lower than the ratio of tryptamine:tryptophan in the body of a control group of animals. In some embodiments, the decrease of tryptamine:tryptophan ratio is at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, or at least 40%. In some embodiment, the test group of animals is fed with a group of feed additives comprising one or more of N-acetyl-muramidase, and protease.

It has been also demonstrated that tryptamine produced by a gut microbe was able to accelerate the whole gut transit (Bhattarai et al, 2018), therefore being able to influence nutrient absorption. Reduction of tryptamine is therefore favorable for increased animal performance.

It has been observed in the present invention that increase of ratios of kynurenine:tryptophan, melatonin:tryptophan, and peripheral serotonin and decrease of ratio of tryptamine:tryptophan are caused by adding a select number of feed additives to the feed of production animals. In a preferred embodiment, the gut health enzyme is N-acetyl-muramidase. In a specific embodiment, the N-acetyl-muramidase is made by DSM Nutritional Products LLC in the commercial name of Balancius. In another preferred embodiment, the protease is made by DSM Nutritional Products LLC in the commercial name of Ronozyme ProAct. In order to produce the health benefits described in this application, a suitable amount of enzyme is required. Such suitable amount is based on the type of animal and its stage of growth and can be determined by experiments. However, a minimal amount of enzyme is required in order to obtain the health benefits. In one embodiment, the enzyme (N-acetyl-muramidase, or protease) is at least 25 g/tone of the feed. In another embodiment. The enzyme is at least 50 g/1000 kg of the feed. An optimal range of concentration which suits best for the present invention has been determined by the inventors of the present application. In some embodiments, the enzyme is between 25-50 g/1000 kg, 50-100 g/1000 kg, 100-200 g/1000 kg, 200-500 g/1000 kg or 500-1000 g/1000 kg of the feed. In a preferred embodiment, the enzyme is between 50-220 g/1000 kg of the feed.

N-acetyl-muramidase: The N-acetyl-muramidase Balancius is characterized in that it is selected from the group consisting of: (a) a polypeptide having at least 80%, e.g., at least 85%, at least 90%, at least 95%, or 100% sequence identity to any one of SEQ ID NOs: 1-71; (b) a variant of a polypeptide having any one of SEQ ID NOs: 1-71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions; (c) a polypeptide comprising the polypeptide of (a) or (b) and a N-terminal and/or C-terminal extension of between 1 and 10 amino acids; and/or (d) a fragment of a polypeptide of (a) or (b) having muramidase activity and having at least 90% of the length of the mature polypeptide. An enzyme referred to as N-acetyl-muramidase Balancius or lysozyme Balancius or muramidase Balancius, or N-acetylmuramide glycanhydrolase Balancius can be produced as described in Example 2 of WO 2019/121937 A1. N-acetyl-muramidase activity can be determined as described in Example 1 of WO 2019/121937 A1.

Protease: The protease Ronozyme ProAct is a serine protease obtained or obtainable from Nocardiopsis sp.. In particular, it can be characterized in that it is derived from Nocardiopsis sp. NRRL 18262, and/or from Nocardiopsis alba (taxonomy based on Berge's Manual of Systematic Bacteriology, 2nd edition, 2000, Springer (preprint: Road Map to Bergey's)). It can also be characterized in that it is an acid-stable serine protease obtained or obtainable from Nocardiopsis dassonvillei subsp. dassonvillei DSM 43235 (A1918L1), Nocardiopsis prasina DSM 15649 (NN018335L1), Nocardiopsis prasina (previously alba) DSM 14010 (NN18140L1), Nocardiopsis sp. DSM 16424 (NN018704L2), Nocardiopsis alkaliphila DSM 44657 (NN019340L2) and Nocardiopsis lucentensis DSM 44048 (NN019002L2), as well as homologous proteases therefrom. In general, the term serine protease refers to serine peptidases and their clans as defined in the Handbook of Proteolytic Enzymes, A. J. Barrett, N. D. Rawlings, J. F. Woessner (eds), Academic Press (1998). In the 1998 version of this handbook, serine peptidases and their clans are dealt with in chapters 1-175. Serine proteases may be defined as peptidases in which the catalytic mechanism depends upon the hydroxyl group of a serine residue acting as the nucleophile that attacks the peptide bond. Examples of serine proteases for use according to the invention are proteases of Clan SA, e. g. Family S2 (Streptogrisin), e. g. Sub-family S2A (alpha-lytic protease), as defined in the above Handbook.

In addition or alternatively, the protease Ronozyme ProAct can be characterized in that it is (a) a polypeptide having a sequence identity of at least 70% to any one of SEQ ID NOs 72-76; (b) a variant of any one of SEQ ID NOs: 72-76, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions; (c) a polypeptide comprising the polypeptide of (a) or (b) and a N-terminal and/or C-terminal His-tag and/or HQ-tag; (d) a polypeptide comprising the polypeptide of (a) or (b) and a N-terminal and/or C-terminal extension of up to 10 amino acids, e.g. 1, 2, 3, 4, 5, 6, 7, 8, 9 or 10 amino acids; or (e) a fragment of the polypeptide of (a) or (b) having protease activity and having at least 90% of the length of the mature polypeptide.

Protease activity can be measured using any assay, in which a substrate is employed, that includes peptide bonds relevant for the specificity of the protease in question. Examples of protease substrates are casein, and pNA-substrates, such as Suc-AAPF-pNA (available e.g. from Sigma S-7388). Another example is Protazyme AK (azurine dyed crosslinked casein prepared as tablets by Megazyme T-PRAK). Example 2 of WO 01/58276 describes suitable protease assays. A preferred assay is the Protazyme assay of Example 2D (the pH and temperature should be adjusted to the protease in question as generally described previously).

The term “protease” is defined herein as an enzyme that hydrolyses peptide bonds. It includes any enzyme belonging to the EC 3.4 enzyme group (including each of the thirteen subclasses thereof http://en.wikipedia.org/wiki/Category:EC_3.4). The EC number refers to Enzyme Nomenclature 1992 from NC-IUBMB, Academic Press, San Diego, California, including supplements 1-5 published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively. The term “subtilases” refer to a sub-group of serine protease according to Siezen et al., Protein Engng. 4 (1991) 719-737 and Siezen et al. Protein Science 6 (1997) 501-523. Serine proteases or serine peptidases is a subgroup of proteases characterized by having a serine in the active site, which forms a covalent adduct with the substrate. Further, the subtilases (and the serine proteases) are characterized by having two active site amino acid residues apart from the serine, namely a histidine and an aspartic acid residue. The subtilases may be divided into 6 sub-divisions, i.e. the Subtilisin family, the Thermitase family, the Proteinase K family, the Lantibiotic peptidase family, the Kexin family and the Pyrolysin family.

A protease referred to herein may not only be natural or wildtype proteases, but also any mutants, variants, fragments etc. thereof exhibiting protease activity, as well as synthetic proteases, such as shuffled proteases, and consensus proteases. Such genetically engineered proteases can be prepared as is generally known in the art, e. g. by Site-directed Mutagenesis, by PCR (using a PCR fragment containing the desired mutation as one of the primers in the PCR reactions), or by Random Mutagenesis. The preparation of consensus proteins is described in e. g. EP 0 897 985.

Such non-wildtype proteases may be based on protease(s) derived from Nocardiopsis sp. NRRL 18262, and Nocardiopsis alba and have at least 60, 65, 70, 75, 80, 85, 90, or at least 95% amino acid identity but not 100% to a wildtype protease. For calculating percentage identity, any computer program known in the art can be used. Examples of such computer programs are the Clustal V algorithm (Higgins, D. G., and Sharp, P. M. (1989), Gene (Amsterdam), 73, 237-244; and the GAP program provided in the GCG version 8 program package (Program Manual for the Wisconsin Package, Version 8, Genetics Computer Group, 575 Science Drive, Madison, Wisconsin, USA 53711) (Needleman, S. B. and Wunsch, C. D., (1970), Journal of Molecular Biology, 48, 443-453. In a particular embodiment, the protease referred to herein may be both, acid-stable and thermostable. The term “thermostable” means for proteases referred to herein to have a temperature optimum is at least 50° C., 52° C., 54° C., 56° C., 58° C., 60° C., 62° C., 64° C., 66° C., 68° C., or at least 70° C.

In some embodiments, the invention relates to a use of feed enzymes (in particular N-acetyl-muramidase, and/or protease) in a diet for feeding to a group of animals a) for improving the health of said group of production animals kept in a confined space, comprising increasing the ratio of kynurenine:tryptophan in the body of said group of animals, wherein the ratio of kynurenine:tryptophan in the digestive system of said group of animals is increased for at least 10% higher than the ratio of kynurenine:tryptophan in the body of a control group of animals which are fed with the same diet except for said feed additives; b) for improving the health of said group of production animals kept in a confined space, comprising increasing the ratio of peripheral serotonin:tryptophan in the digestive system of said group of animals, wherein the ratio of peripheral serotonin:tryptophan in the brain of said group of animals is increased for at least 20% higher than the ratio of peripheral serotonin:tryptophan in the digestive system of a control group of animals which are fed with the same diet except for said feed additives; c) for improving the health of said group of production animals kept in a confined space, comprising decreasing the ratio of tryptamine:tryptophan in the digestive system of said group of animals, wherein the ratio of tryptamine:tryptophan in the digestive system of said group of animals is decreased for at least 20% lower than the ratio of tryptamine:tryptophan in the digestive system of a control group of animals which are fed with the same diet except for said feed additives; and/or d) for improving the health of said group of production animals kept in a confined space, comprising increasing the ratio of melatonin:tryptophan in the digestive system of said group of animals, wherein the ratio of melatonin:tryptophan in the digestive system of said group of animals is increased for at least 10% higher than the ratio of melatonin:tryptophan in the digestive system of a control group of animals which are fed with the same diet except for said group of feed additives. In a preferred embodiment, the invention relates to a use of N-acetyl-muramidase and/or protease in a diet for feeding to a group of animals a) for improving the health of said group of production animals kept in a confined space, comprising increasing the ratio of kynurenine:tryptophan in the body of said group of animals, wherein the ratio of kynurenine:tryptophan in the digestive system of said group of animals is increased for at least 10% higher than the ratio of kynurenine:tryptophan in the body of a control group of animals which are fed with the same diet except for said feed additives; b) for improving the health of said group of production animals kept in a confined space, comprising increasing the ratio of peripheral serotonin:tryptophan in the digestive system of said group of animals, wherein the ratio of peripheral serotonin:tryptophan in the brain of said group of animals is increased for at least 20% higher than the ratio of peripheral serotonin:tryptophan in the digestive system of a control group of animals which are fed with the same diet except for said feed additives; c) for improving the health of said group of production animals kept in a confined space, comprising decreasing the ratio of tryptamine:tryptophan in the digestive system of said group of animals, wherein the ratio of tryptamine:tryptophan in the digestive system of said group of animals is decreased for at least 20% lower than the ratio of tryptamine:tryptophan in the digestive system of a control group of animals which are fed with the same diet except for said feed additives; and/or d) for improving the health of said group of production animals kept in a confined space, comprising increasing the ratio of melatonin:tryptophan in the digestive system of said group of animals, wherein the ratio of melatonin:tryptophan in the digestive system of said group of animals is increased for at least 10% higher than the ratio of melatonin:tryptophan in the digestive system of a control group of animals which are fed with the same diet except for said group of feed additives. Preferably, the N-acetyl-muramidase is selected from the group consisting of: (a) a polypeptide having at least 80% sequence identity to any one of SEQ ID NOs: 1-71; (b) a variant of a polypeptide having any one of SEQ ID NOs: 1-71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions; (c) a polypeptide comprising the polypeptide of (a) or (b) and a N-terminal and/or C-terminal extension of between 1 and 10 amino acids; and (d) a fragment of a polypeptide of (a) or (b) having muramidase activity and having at least 90% of the length of the mature polypeptide; and preferably the protease is selected from the group consisting of: (a′) a polypeptide having a sequence identity of at least 70% to any one of SEQ ID NOs 72-76; (b′) a variant of any one of SEQ ID NOs: 72-76, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions; (c′) a polypeptide comprising the polypeptide of (a′) or (b′) and a N-terminal and/or C-terminal His-tag and/or HQ-tag; (d′) a polypeptide comprising the polypeptide of (a′) or (b′) and a N-terminal and/or C-terminal extension of up to 10 amino acids, e.g. 1, 2, 3, 4, 5, 6, 7, 8, 9 or 10 amino acids; and (e′) a fragment of the polypeptide of (a′) or (b′) having protease activity and having at least 90% of the length of the mature polypeptide.

### Type of Animal

The method of the present invention is applicable to production animals in general. In one embodiment, the method of the present invention is applicable to poultry.

The above mentioned feed additives may be provided to any suitable animal. In some embodiments, the animal is monogastric. It is generally understood that a monogastric animal has a single-chambered stomach. In other embodiments, the animal is a ruminant. It is generally understood that a ruminant has a multi-chambered stomach. In some embodiments, the animal is a ruminant in the pre-ruminant phase. Examples of such ruminants in the pre-ruminant phase include nursery calves.

In some embodiments, the animal is a poultry (e.g. chicken, turkey), seafood (e.g. shrimp), sheep, cow, cattle, buffalo, bison, pig (e.g. nursery pig, grower/finisher pig), cat, dog, rabbit, goat, guinea pig, donkey, camel, horse, pigeon, ferret, gerbil, hamster, mouse, rat, bird, or human.

In some embodiments, the animal is livestock. In some embodiments, the animal is a companion animal. In some embodiments, the animal is poultry. Examples of poultry include chicken, duck, turkey, goose, quail, or Cornish game hen. In one variation, the animal is a chicken. In some embodiments, the poultry is a layer hen, a broiler chicken, or a turkey.

In other embodiments, the animal is a mammal, including, for example, a cow, a pig, a goat, a sheep, a deer, a bison, a rabbit, an alpaca, a llama, a mule, a horse, a reindeer, a water buffalo, a yak, a guinea pig, a rat, a mouse, an alpaca, a dog, or a cat. In one variation, the animal is a cow. In another variation, the animal is a pig. In another variation, the animal is a sow.

### Administration of Feed Additives

In some embodiments, administration comprises providing the feed additives described herein to an animal such that the animal may ingest the feed additives at will. In such embodiments, the animal ingests some portion of the feed additives.

The feed additives described herein may be provided to the animal on any appropriate schedule. In some embodiments, the animal is the feed additives described herein on a daily basis, on a weekly basis, on a monthly basis, on an every other day basis, for at least three days out of every week, or for at least seven days out of every month.

In some embodiments, the feed additives described herein is administered to the animal multiple times in a day. For examples, in some embodiments, the feed additives described herein is administered to the animal at least 1, 2, 3, 4, 5, 6, 7, 8, 9, or 10 times a day. In some embodiments, the nutritional composition, the feed additives described herein is administered to the animal at most 1, 2, 3, 4, 5, 6, 7, 8, 9, or 10 times a day.

In some embodiments, the feed additives described herein is administered to the animal multiple times in a day. For examples, in some embodiments, the feed additives described herein is administered to the animal at least 1, 2, 3, 4, 5, 6, 7, 8, 9, or 10 times a week. In some embodiments, the nutritional composition, the feed additives described herein is administered to the animal at most 1, 2, 3, 4, 5, 6, 7, 8, 9, or 10 times a week. In some embodiments, the feed additives described herein is administered to the animal every day, every other day, every 3 days, every 4 days, every week, every other week, or every month.

In some embodiments, the animal is the feed additives described herein during certain diet phases. For example, some animals are provided a starter diet between 0 to 14 days of age. In other embodiments, an animal is provided a grower diet between 15 to 28 days of age, between 15 to 35 days of age, or between 15 to 39 days of age. In still other embodiments, an animal is provided a finisher diet between 29 to 35 days of age, between 36 to 42 days of age, or between 40 to 46 days of age.

In certain embodiments, the feed additives described herein is provided to the animal during the starter diet phase, the grower diet phase, or the finisher diet phase, or any combinations thereof. In certain embodiments, the animal is poultry, and the poultry is provided a starter diet between 0 to 15 days of age, a grower diet between 16 to 28 days of age, and a finisher diet between 29 to 35 days of age. In other embodiments, the animal is poultry, and the poultry is provided a starter diet between 0 to 14 days of age, a grower diet between 15 to 35 days of age, and a finisher diet between 36 to 42 days of age. In still other embodiments, the animal is poultry, and the poultry is provided a starter diet between 0 to 14 days of age, a grower diet between 15 to 39 days of age, and a finisher diet between 20 to 46 days of age.

In some embodiments, the feed additives described herein is provided to the poultry during the starter diet phase, the grower diet phase, or the finisher diet phase, or any combinations thereof.

The feed additives described herein may be fed to individual animals or an animal population. For example, in one variation where the animal is poultry, the feed additives described herein may be fed to an individual poultry or a poultry population.

The feed additives described herein may be provided to an animal in any appropriate form, including, for example, in solid form, in liquid form, or a combination thereof. In certain embodiments, the feed additives described herein is a liquid, such as a syrup or a solution. In other embodiments, the feed additives described herein is a solid, such as pellets or powder. In yet other embodiments, the feed additives described herein may be fed to the animal in both liquid and solid components, such as in a mash.

### EXAMPLES

### Example 1

### Preparation of Poultry Feed

Control Feed was a commercial U.S. corn-soy starter poultry feed. Treated Feed was a commercial U.S. corn-soy starter poultry feed containing 200 ppm of a Ronozyme ProAct protease preparation. For the treated diet, the protease preparation was provided in a powder form and adding the powder to the mixer using a micro-ingredient balance prior to pelleting.

For the control diet, the same commercial U.S. corn-soy starter poultry feed was used without the addition of any Ronozyme ProAct protease.

The above industry-standard corn-soy poultry feeds were manufactured according to industry practices. In the treated diet, Ronozyme ProAct protease was supplemented to the control diet ay 200 ppm. A three-phase feeding program with the control diet and treated diet were conducted.

### Example 2

Trial with Ronozyme ProAct Protease Treated Diet and Control Diet

Ross 308 male broilers were placed randomly into floor pens constructed in a poultry house, with 40 birds per pen and a stocking density of about 1 square foot per bird. Pens were assigned randomly to treatment groups, with 3 statistical replicates per treatment and pen as the experimental unit. For each pen, the bedding consisted of built-up litter top-dressed with fresh wood shavings.

A standard commercial environmental and lighting program was employed. Starter diets were fed as crumbles, finisher diets were fed as pellets. All diets were provided ad libitum via automatic feeders in each pen, and on feeder trays from day one until day 7.

Water was provided ad libitum from a nipple drinking line. Animals and housing facilities were inspected daily, including recording the general health status, feed consumption, water supply and temperature of the facility. Any mortalities were recorded daily. The total mass of consumed feed was recorded for each pen. Weight gain and FCR were then determined for each pen according to standard practices. FCR was corrected for mortality and adjusted to a common body weight.

### Example 3

### Cecal Microbiome Sampling and Metabolites Measurements

On day 42, eight birds from the group fed with control diet and eight birds from the group fed with treated diet were selected. The live weight of each sampled bird was recorded. Each sampled bird was then euthanized via cervical dislocation followed by extraction of the cecal using standard veterinary methods. Following dissection, cecal contents were transferred to 5 mL conical tubes, the weight of the cecal contents was recorded, and the contents were flash frozen to −80° C. A small ileal tissue sample was collected by resection from the intestinal wall, followed by prompt treatment with RNA-polymerase inhibitor.

Entire metabolomics procedure was performed at Metabolon, Inc. (North Carolina, USA). Samples were extracted with methanol under strong shaking to precipitate protein and dissociate small molecules bound or trapped into proteins, then centrifuged. The resulting extracts were divided into five fractions. Two fractions were analyzed by two separate reverse phase (RP)/UPLC-MS/MS methods using positive ion mode electrospray ionization (ESI). One fraction was analyzed by RP/UPLC-MS/MS using negative ion mode ESI. One was analyzed by HILIC/UPLC-MS/MS using negative ion mode ESI. One fraction was preserved as backup sample. All five samples were briefly removed of organic solvents by TurboVap.

Observation of Tryptophan level in Broiler's Gut

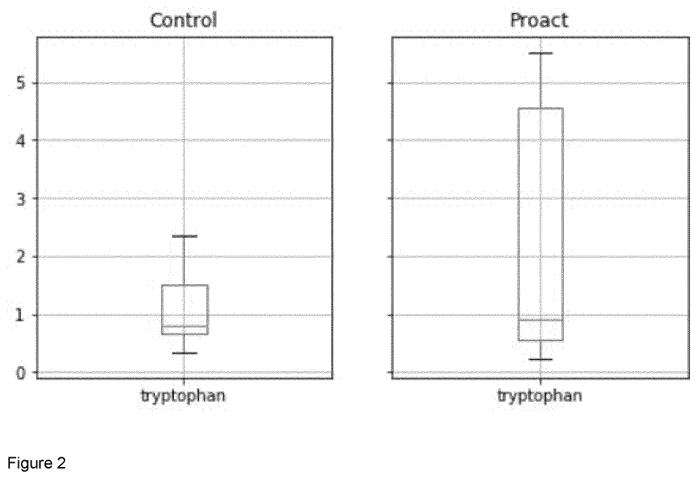

Statistical analysis of study outcomes was performed in the R statistical computing language [R version 3.4.4 (2018-03-15)]. The level of tryptophan in the gut of the broilers in the Ronozyme ProAct protease treated group and the untreated control group were measured. Broilers treated with Ronozyme ProAct protease showed higher level tryptophan in the gut. See FIG. 2.

### Observation of Metabolites/Tryptophan Ratio in Broiler's Guts

As shown in FIG. 1, it is known that tryptophan is further catabolized into different metabolites via different pathways. Therefore, the ratio of tryptophan metabolites against tryptophan, for example, anthranilate:tryptophan, kynurenine:tryptophan, quinolinate:tryptophan, serotonin:tryptophan, and tryptamine:tryptophan, was measured. It was observed that all these tryptophan metabolites:tryptophan ratio, have increased in the broilers treated with Ronozyme ProAct protease when comparing to the untreated control group, except tryptamine:tryptophan ratio that has decreased. This result suggests that the flux in the kynurenine pathway, the serotonin pathway are increased, the one in the tryptamine pathway has decreased.

The numerical data of tryptophan metabolites:tryptophan ratio is shown in Table 1. It was observed that metabolites:tryptophan ratio of the treated group is more than 10% higher than the untreated group.

TABLE 1

| | Ronozyme | Untreated |
|---|---|---|
| metabolites:tryptophan | ProAct protease | control |
| ratio in fecal sample | treated group | group |
| kynurenine:tryp ratio | 1.59 | 0.91 |
| peripheral serotonin:tryp ratio | 1.53 | 0.61 |
| melatonin:tryp ratio | 1.12 | 0.51 |
| tryptamine:tryp ratio | 0.134 | 0.22 |

### Example 4

Welfare Status of Broiler Chickens Fed with Ronozyme ProAct Protease

### Reduction of Social Disturbance Behaviors

Social behavior analysis is performed by five independent observers from study days 35 to 42. It is observed that the incidents of feather pecking per chicken per 10 minutes in the Ronozyme ProAct protease treated group is at least 10% lower than the untreated control group.

The changes in serum insulin and glucagon levels are measured in the Ronozyme ProAct protease treated group and the control group. The concentrations of both serum insulin and glucagon increase to a higher level in the Ronozyme ProAct protease treated group than that of the control group. The experimental results are consistent with the earlier observation that the ratios of serotonin:tryp and melatonin:tryp are higher in the Ronozyme ProAct protease treated group than that of the control group.

### Reduction of Illuminated Period on Broilers

In this study, the same Ross 308 male broilers are subjected to two different illuminated conditions. Group I of the first cohort of 20-day old broilers is subjected to 1 hour darkness and 23 hours light condition for 14 days. Group II of the second cohort of 20-day old broilers is subjected to 8 hours of darkness and 16 hours of light condition. The light intensity is 15 lx. It is observed that Group II broilers produce about 12% higher concentration of serum insulin and glucagon levels than the Group I broilers. This finding indicates that photoperiod has a specific effect on the synthesis of insulin and glucagon. When compared with the result of the early study on the effect of Ronozyme ProAct protease on the synthesis of insulin and glucagon, it suggests that feeding Ronozyme ProAct protease to broilers may produce a similar effect of prolonging the daylight period to the broilers. This may improve the welfare of the birds by reducing the unnaturally prolonged illuminated condition back to the regular photoperiod rhythm.

1. A method for improving the health of a group of production animals kept in a confined space, the method comprising increasing the ratio of kynurenine:tryptophan in the body of said group of animals by feeding said group of production animals one of more of the following feed additives: N-acetyl-muramidase, and protease, wherein the ratio of kynurenine:tryptophan in the digestive system of said group of animals is increased for at least 10% higher than the ratio of kynurenine:tryptophan in the body of a control group of animals which are fed with the same diet except for said feed additives;
preferably wherein the N-acetyl-muramidase is selected from the group consisting of: (a) a polypeptide having at least 80% sequence identity to any one of SEQ ID NOs: 1-71; (b) a variant of a polypeptide having any one of SEQ ID NOs: 1-71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions; (c) a polypeptide comprising the polypeptide of (a) or (b) and a N-terminal and/or C-terminal extension of between 1 and 10 amino acids; and (d) a fragment of a polypeptide of (a) or (b) having muramidase activity and having at least 90% of the length of the mature polypeptide; and
preferably wherein the protease is selected from the group consisting of: (a′) a polypeptide having a sequence identity of at least 70% to any one of SEQ ID NOs 72-76; (b′) a variant of any one of SEQ ID NOs: 72-76, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions; (c′) a polypeptide comprising the polypeptide of (a′) or (b′) and a N-terminal and/or C-terminal His-tag and/or HQ-tag; (d′) a polypeptide comprising the polypeptide of (a′) or (b′) and a N-terminal and/or C-terminal extension of up to 10 amino acids, e.g. 1, 2, 3, 4, 5, 6, 7, 8, 9 or 10 amino acids; and (e′) a fragment of the polypeptide of (a′) or (b′) having protease activity and having at least 90% of the length of the mature polypeptide.

2. The method of claim 1, wherein said ratio of kynurenine: tryptophan is measured in the feces or blood of said animals.

3. The method of claim 2, wherein said improving the health of said group of production animals comprises providing one of more of the following benefits to said group of production animals: improving the welfare of said group of production animals, decreasing systemic inflammation of said group of production animals, decreasing local inflammation of said group of production animals, and reducing the light regimen into the daily circadian rhythm of said group of production animals.

4. The method of claim 3, wherein said improvement of welfare comprises reducing social disturbance among said group of production animals.

5. The method of claim 3, wherein said improvement of welfare comprises reducing feather pecking among said group of production animals.

6. The method of claim 1, wherein the concentration of said N-acetyl-muramidase, or protease is between 50 and 1000 g/tonne of the feed to be given to the group of production animals.

7. The method of claim 1, wherein said production animals are: broiler chickens, turkeys, ducks, layers, piglets, grower pigs, finisher pigs, and sows.

8. A method for improving the health of a group of production animals kept in a confined space, the method comprising increasing the ratio of peripheral serotonin:tryptophan in the digestive system of said group of animals by feeding said group of production animals one of more of the following feed additives: N-acetyl-muramidase, and protease, wherein the ratio of peripheral serotonin:tryptophan in the brain of said group of animals is increased for at least 20% higher than the ratio of peripheral serotonin:tryptophan in the digestive system of a control group of animals which are fed with the same diet except for said feed additives;
preferably wherein the N-acetyl-muramidase is selected from the group consisting of: (a) a polypeptide having at least 80% sequence identity to any one of SEQ ID NOs: 1-71; (b) a variant of a polypeptide having any one of SEQ ID NOs: 1-71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions; (c) a polypeptide comprising the polypeptide of (a) or (b) and a N-terminal and/or C-terminal extension of between 1 and 10 amino acids; and (d) a fragment of a polypeptide of (a) or (b) having muramidase activity and having at least 90% of the length of the mature polypeptide; and
preferably wherein the protease is selected from the group consisting of: (a′) a polypeptide having a sequence identity of at least 70% to any one of SEQ ID NOs 72-76; (b′) a variant of any one of SEQ ID NOs: 72-76, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions; (c′) a polypeptide comprising the polypeptide of (a′) or (b′) and a N-terminal and/or C-terminal His-tag and/or HQ-tag; (d′) a polypeptide comprising the polypeptide of (a′) or (b′) and a N-terminal and/or C-terminal extension of up to 10 amino acids, e.g. 1, 2, 3, 4, 5, 6, 7, 8, 9 or 10 amino acids; and (e′) a fragment of the polypeptide of (a′) or (b′) having protease activity and having at least 90% of the length of the mature polypeptide.

9. The method of claim 8, wherein said ratio of peripheral serotonin:tryptophan is measured in the feces of said animals.

10. The method of claim 9, wherein said improving the health of said group of production animals comprises providing one of more of the following benefits to said group of production animals: improving the welfare of said group of production animals, decreasing systemic inflammation of said group of production animals, decreasing local inflammation of said group of production animals, and reducing the light regimen into the daily circadian rhythm of said group of production animals.

11. The method of claim 9, wherein said improvement of welfare comprises reducing social disturbance among said group of production animals.

12. The method of claim 9, wherein said improvement of welfare comprises reducing feather pecking among said group of production animals.

13. The method of claim 9, wherein said improvement of welfare comprises restoring the natural photoperiod of said group of production animals.

14. The method of claim 13, wherein said natural photoperiod comprises at least 8 hours of darkness.

15. The method of claim 8, wherein the concentration of said N-acetyl-muramidase, or protease is between 50 and 1000 g/tonne of the feed to be given to the group of production animals.

16. The method of claim 8, wherein said production animals are: broiler chickens, turkeys, ducks, layers, piglets, grower pigs, finisher pigs, and sows.

17. A method for improving the health of a group of production animals kept in a confined space, the method comprising decreasing the ratio of tryptamine:tryptophan in the digestive system of said group of animals by feeding said group of production animals one of more of the following feed additives: N-acetyl-muramidase, and protease, wherein the ratio of tryptamine:tryptophan in the digestive system of said group of animals is decreased for at least 20% lower than the ratio of tryptamine:tryptophan in the digestive system of a control group of animals which are fed with the same diet except for said feed additives;
preferably wherein the N-acetyl-muramidase is selected from the group consisting of: (a) a polypeptide having at least 80% sequence identity to any one of SEQ ID NOs: 1-71; (b) a variant of a polypeptide having any one of SEQ ID NOs: 1-71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions; (c) a polypeptide comprising the polypeptide of (a) or (b) and a N-terminal and/or C-terminal extension of between 1 and 10 amino acids; and (d) a fragment of a polypeptide of (a) or (b) having muramidase activity and having at least 90% of the length of the mature polypeptide; and
preferably wherein the protease is selected from the group consisting of: (a′) a polypeptide having a sequence identity of at least 70% to any one of SEQ ID NOs 72-76; (b′) a variant of any one of SEQ ID NOs: 72-76, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions; (c′) a polypeptide comprising the polypeptide of (a′) or (b′) and a N-terminal and/or C-terminal His-tag and/or HQ-tag; (d′) a polypeptide comprising the polypeptide of (a′) or (b′) and a N-terminal and/or C-terminal extension of up to 10 amino acids, e.g. 1, 2, 3, 4, 5, 6, 7, 8, 9 or 10 amino acids; and (e′) a fragment of the polypeptide of (a′) or (b′) having protease activity and having at least 90% of the length of the mature polypeptide.

18. The method of claim 17, wherein said ratio of tryptamine:tryptophan is measured in the feces of said animals.

19. The method of claim 18, wherein said improving the health of said group of production animals comprises providing one of more of the following benefits to said group of production animals: improving the performance of said group of production animals, improving the welfare of said group of production animals, decreasing systemic inflammation of said group of production animals, decreasing local inflammation of said group of production animals, and reducing the light regimen into the daily circadian rhythm of said group of production animals.

20. The method of claim 19, wherein improving performance of said group of production animals comprises providing one of more of the following benefits to said group of production animals: improving nutrient absorption, reduce gut peristaltic motility, improving vitamin absorption, and improving feed enzymatic processing.

21. The method of claim 19, wherein said improvement of welfare comprises reducing social disturbance among said group of production animals.

22. The method of claim 19, wherein said improvement of welfare comprises reducing feather pecking among said group of production animals.

23. The method of claim 17, wherein the concentration of said N-acetyl-muramidase, or protease is between 50 and 1000 g/tonne of the feed to be given to the group of production animals.

24. The method of claim 17, wherein said production animals are: broiler chickens, turkeys, ducks, layers, piglets, grower pigs, finisher pigs, and sows.

25. A method for improving the health of a group of production animals kept in a confined space, the method comprising increasing the ratio of melatonin:tryptophan in the digestive system of said group of animals by feeding said group of production animals one of more of the following group of feed additives: N-acetyl-muramidase, and protease, wherein the ratio of melatonin:tryptophan in the digestive system of said group of animals is increased for at least 10% higher than the ratio of melatonin:tryptophan in the digestive system of a control group of animals which are fed with the same diet except for said group of feed additives;
preferably wherein the N-acetyl-muramidase is selected from the group consisting of: (a) a polypeptide having at least 80% sequence identity to any one of SEQ ID NOs: 1-71; (b) a variant of a polypeptide having any one of SEQ ID NOs: 1-71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions; (c) a polypeptide comprising the polypeptide of (a) or (b) and a N-terminal and/or C-terminal extension of between 1 and 10 amino acids; and (d) a fragment of a polypeptide of (a) or (b) having muramidase activity and having at least 90% of the length of the mature polypeptide; and
preferably wherein the protease is selected from the group consisting of: (a′) a polypeptide having a sequence identity of at least 70% to any one of SEQ ID NOs 72-76; (b′) a variant of any one of SEQ ID NOs: 72-76, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions; (c′) a polypeptide comprising the polypeptide of (a′) or (b′) and a N-terminal and/or C-terminal His-tag and/or HQ-tag; (d′) a polypeptide comprising the polypeptide of (a′) or (b′) and a N-terminal and/or C-terminal extension of up to 10 amino acids, e.g. 1, 2, 3, 4, 5, 6, 7, 8, 9 or 10 amino acids; and (e′) a fragment of the polypeptide of (a′) or (b′) having protease activity and having at least 90% of the length of the mature polypeptide.

26. The method of claim 25, wherein said ratio of melatonin:tryptophan is measured in the feces or blood of said animals.

27. The method of claim 26, wherein said improving the health of said group of production animals comprises providing one of more of the following benefits to said group of production animals: improving the welfare of said group of production animals, decreasing systemic inflammation of said group of production animals, decreasing local inflammation of said group of production animals, and reducing the light regimen into the daily circadian rhythm of said group of production animals.

28. The method of claim 27, wherein said improvement of welfare comprises reducing social disturbance among said group of production animals.

29. The method of claim 28, wherein said improvement of welfare comprises reducing feather pecking among said group of production animals.

30. The method of claim 25, wherein the concentration of said N-acetyl-muramidase, or protease is between 50 and 1000 g/1000 kg of the feed to be given to the group of production animals.

31. The method of claim 25, wherein said production animals are: broiler chickens, turkeys, ducks, layers, piglets, grower pigs, finisher pigs, and sows.

32. Use of feed enzymes, in particular N-acetyl-muramidase, and/or protease, in a diet for feeding to a group of animals for
a) improving the health of said group of production animals kept in a confined space, comprising increasing the ratio of kynurenine:tryptophan in the body of said group of animals, wherein the ratio of kynurenine:tryptophan in the digestive system of said group of animals is increased for at least 10% higher than the ratio of kynurenine:tryptophan in the body of a control group of animals which are fed with the same diet except for said feed additives;
b) improving the health of said group of production animals kept in a confined space, comprising increasing the ratio of peripheral serotonin:tryptophan in the digestive system of said group of animals, wherein the ratio of peripheral serotonin:tryptophan in the brain of said group of animals is increased for at least 20% higher than the ratio of peripheral serotonin:tryptophan in the digestive system of a control group of animals which are fed with the same diet except for said feed additives;
c) improving the health of said group of production animals kept in a confined space, comprising decreasing the ratio of tryptamine:tryptophan in the digestive system of said group of animals, wherein the ratio of tryptamine:tryptophan in the digestive system of said group of animals is decreased for at least 20% lower than the ratio of tryptamine:tryptophan in the digestive system of a control group of animals which are fed with the same diet except for said feed additives; and/or
d) improving the health of said group of production animals kept in a confined space, comprising increasing the ratio of melatonin:tryptophan in the digestive system of said group of animals, wherein the ratio of melatonin:tryptophan in the digestive system of said group of animals is increased for at least 10% higher than the ratio of melatonin:tryptophan in the digestive system of a control group of animals which are fed with the same diet except for said group of feed additives.


SEQUENCE LISTING (76 sequences)

SEQ ID NO: 1 - protein, 208 aa, Acremonium alcalophilum
RIPGFDISGWQPTTDFARAYANGDRFVYIKATEGTTFKSSAFSRQYTGATQNGFIRGAYHFAQPAASSGAAQARYFASNGGGWSKDGITLPGALDIEYNPNGATCYGLSQSAMVNWIEDFVTTYHGITSRWPVIYTTTDWWTQCTGNSNRFANRCPLWIARYASSVGTLPNGWGFYTFWQYNDKYPQGGDSNWFNGDASRLRALANGD

SEQ ID NO: 2 - protein, 213 aa, Acremonium alcalophilum
SPIRRRIPGFDISGWQPTTDFARAYANGDRFVYIKATEGTTFKSSAFSRQYTGATQNGFIRGAYHFAQPAASSGAAQARYFASNGGGWSKDGITLPGALDIEYNPNGATCYGLSQSAMVNWIEDFVTTYHGITSRWPVIYTTTDWWTQCTGNSNRFANRCPLWIARYASSVGTLPNGWGFYTFWQYNDKYPQGGDSNWFNGDASRLRALANGD

SEQ ID NO: 3 - protein, 218 aa, Aspergillus fumigatus
LPSQPEARATTVQGFDISNHQKSVNFEAAKKDGAQFVMIKATEGTTYKDTVFNSHYTGATKAGLLRGGYHFARPDKSTGSTQAKFFLKNGGGWSDDNRTLPGMLDIEYNPYGATCYGLSHSQMVAWIHDFVNEYHHATSRWPMIYTTADWWNRCTGNAKGFGDKCPLVLAAYSSSPPKTIPGDWKTWTIWQNSDKYKHGGDSDKFNGPMTQLRKLASG

SEQ ID NO: 4 - protein, 208 aa, Trichoderma reesei
TVPGFDISHYQATVDFAKAYADGARFVIIKATEGTTYTDPSFSDHYTKATNAGFIRGGYHFAQPASSSGAAQANYFLKHGGGWSADGITLPGMLDLEYAPSGDSCYGLSASAMVSWINDFVNTYHAATTQYPLIYTSTSWWQLCTGNNGSFGSKSPLVIARYASSVGALPNGWSVYTIWQNSDASPWGGDNDIFNGNLAQLQKIARGS

SEQ ID NO: 5 - protein, 215 aa, Trametes cinnabarina
SPTPEKRANPKGIDVSAYQPNINWSTVKANGISFAYIKATEGTTYTNPDFSSQYTGATNAGLIRGGYHFAHPDSSSGATQAKYFLAHGGGWTSDGITLPGALDIEYNPSGAECYGLSASAMVSWIKDFSNTYHSSTGVYPVIYTTTDWWTTCTGNSAAFASTNPLWIARYASSIGTLPAGWSYTTFWQYADSGPNPGDQDEFNGSMAGLKQLALG

SEQ ID NO: 6 - protein, 207 aa, Sporormia fimetaria
AVPGFDISHYQSSVNYAGAYSSGARFVIIKATEGTTYIDPKFSDHYIGATNAGLIRGAYHFARPAASTGAAQANYFVSHGGGWSADGITLPGMLDMEYGSTSACHGLSQSAMVTWITSFVNQYNSLTGRYPMIYTTADWWQTCTGNSAAFNTKSPLVLARYSSSAGTVPGGWPYYTIWQFNDAYAYGGDSDTFNGDLAGLKRLAKGS

SEQ ID NO: 7 - protein, 201 aa, Poronia punctata
AVQGFDISHYQSSVNFGAAKSSGAQFVIIKATEGTSYTDPSFSSHYTGATNAGLIRGGYHFAHLDSSSGAAQAKYFLAHGGGWSGDGITLPGMLDLEGSCVLSASATVSWIKDFSNTYHSSTGVYPLIYTNPSWWSSCTGNSKAFIDTNPLVLARYASSAGTPPGGWPYYTIWQYNDAYKYGGDSDVFNGDLAGLKRLAKG

SEQ ID NO: 8 - protein, 201 aa, Poronia punctata
QVQGFDISSYQPSVDFAGAYADGARFVIIKATEGTGYIDPTFSDHYVGATNAGLLRGGYHYAHLDSTSGATQAQYFLANGGGWSGDGITLPGMLDLEGDCVLSAADAVAWIKDFSDTYHASTGVYPLLYTNPSWWASCTGDSSAFIDTNPLVLAHYADAAGTPPGGWPFYSFWQYNDAYPYGGDSEVWNGDMDGLLRLASG

SEQ ID NO: 9 - protein, 203 aa, Lecanicillium sp. WMM742
VDSSSEVSVAIYKKALGQGFTRAIFRGYQEACSQGGRVDPTFVPSYKNAVAAGYKDFDAYFFPCTGKTNKCKPYAAQLAELLDTIKGQKLAIRRIWLDIETDRVCNPFDYGAQGNLAEAKKLVAAFRDAKLDWGIYTSPTQWETIFGAKTVELAKDVPLWFAKFDNVETLELKTPFGGWTKADAKQYTDQSASNKFDLNVFSA

SEQ ID NO: 10 - protein, 208 aa, Lecanicillium sp. WMM742
TVQGFDVSGYQPTVNWGAAYSSGARFVMIKATEGTGYISSSFGSQYPGATNAGFIRGGYHFALPDRSSGSAQADYFLAHGGGWSGDGITLPGMLDIEYNPYGATCYGLSQGAMVNWISDFVEHYKARTTQYPIIYTTTDWWKTCTGNSPAFGQKCPLSLARYSSSVGEIPNGWPFQTFWQNSDKYAYGGDSQIFNGAYSQLQKIARGG

SEQ ID NO: 11 - protein, 207 aa, Onygena equina
AVPGIDVSGYQGNVNWANVANAGKKFAYVKATEHTNYINPYFAQQYNGAYNQGIIRGAYHYAHPNGASGASQANYFLAHGGGWSADGKTLPGAVDLEYGPNGSTCWGISQSAMIAWIRDFSNTYRAKTGRPPVIYTSTSWWKTCTGNYGGFGNDNPLWIARYSSTVGELPAGWPFHSIWQNNDNSGVGGDGDIWNGDLAGLQRFAKG

SEQ ID NO: 12 - protein, 208 aa, Purpureocillium lilacinum
AVKGFDISHYQPNVDFAKAYADGARFVMIKATEGTTYTDPSFSSHYTGATKAGFIRGGYHFARPASSSGAAQAKYFIAHGGGWSKDGITLPGMLDMEYQSSSSACGGLSQSAMVSWINDFVNTYHAATGVYPLIYTSTSWWTQCTGNSAAFGSKCPLVVARYASSVGTLPAGWGFYTFWQYSDAAPWGGDADTFNGDITALKKIANAG

SEQ ID NO: 13 - protein, 207 aa, Trichobolus zukalii
AVPGFDISHYQPSVNYAGAYNSGARFVIIKATEGTTYTDPVFSTHYTGATKAGLIRGGYHFARPASSSGSAQADFFFKNGGGWSADGITLPGMLDMEYGSTSSCHGLSQTAMVNWISDFVNRYKTLSGRYPMIYTGYYWWVECTGNSNKFATTCPLVLARYSSSVGEIPGGWGYQTIWQFNDKYAYGGDSDSFNGSLDRLKALAKGT

SEQ ID NO: 14 - protein, 207 aa, Penicillium citrinum
LIHAVDSSSEVSVDIYKKALSEGFSRAIFRGYQEACSQGGRVDPTFLPSYKNAQTAGYKDFDAYFFPCTGSGNKCKPYDVQIGELVDAIKKNNMAIRRIWVDFEKDKTCNPFNWDPKRNIDEAKRIIGAVRKTKFDFGVYTSATQWTSIFGSKDVVLANDVPLWFAKFDNVENLDLAQPFGGWTKADGKQYTDKSASKKFDLNVFSA

SEQ ID NO: 15 - protein, 207 aa, Cladorrhinum bulbillosum
AVQGFDISHYQSSVNFQAAYNSGARFVIIKATEGTTYIDPKFSSHYTGATNAGLIRGGYHFAHPDSSTGAAQADFFLAHGGGWSGDGITLPGMLDLESVSGKATCFGLSASSMVAWIKSFSDRYHTRTGRYPMLYTNPSWWTTCTGNSNAFVNTNPLVLARYASAPGTIPGGWPYQTIWQNSDSYTYGGDSDIFNGALSGLQKLASG

SEQ ID NO: 16 - protein, 208 aa, Umbelopsis westeae
KLKGLDVSGYQPNVAWSTVKANGASFAYIKATEGTNYKNPSFAQQYNGAYNAGLIRGSYHFAQPSSSTGAAQANYFLAHGGGWSPDGKTLPGALDMEYNPHGSTCYGLSKDAMVKWIKDFSNTYHSATGRYPVIYTTTSWWTTCTGNSAAFGATNPLWIARYSSTAGNLPNGWAFYSFWQNADSGIFPGDQDIWNGDAAALSRMAKGA

SEQ ID NO: 17 - protein, 208 aa, Zygomycetes sp. XZ2655
TLPGLDVSSYQGNVNWGTVASQGAKFAYVKATEGTTYTNPYFASQYDGSYNAGLIRGAYHFAHPDSSSGATQANYFLAHGGGWSADGKTLPGALDIEYNPNGAECYGLSQLAMISWIQDFSNTYHSHTGRYPVIYTTTDWWTTCTGNSAAFGTNNPLWIARYSSSVGTLPAGWGYESFWQKASSGTFPGDQDIWNGDAAGLSRFATGK

SEQ ID NO: 18 - protein, 206 aa, Chaetomium cupreum
TVQGFDISGYQPNVNFAAAYAAGARFVIIKATEGTSYISPSFSSQYTGATNAGFIRGGYHFAHPGASSGTTQADYFIAHGGGWTPDGITLPGMLDLESESSGTCWGLSASAMVAWIKDFSDHYHSRMGVYPLLYTNPSWWEECTGNSNAFVDTNPLVLAHYSSSVGTIPGGWPYETIWQNSDSYAYGGDSDVFNGDLAGLQRLARG

SEQ ID NO: 19 - protein, 207 aa, Cordyceps cardinalis
LIHAVDSSSEVSVDVYKKALAEGFTRAIFRGYQEACSQGGRVDPTFLPSYKNAQKAGYKDFDAYFFPCTGSGNKCKPYAKQIGELVDAIEGNQLAIRRIWIDIETDKVCNAFNWGAEGNIQEAKKLIAAVRGTKRDFGIYTSATQWENIFGSRTVELAKDVPLWFAKFDNVETLELKTPFGGWTKADAKQYTDKSASKKFDLNVFSA

SEQ ID NO: 20 - protein, 216 aa, Penicillium sp. ′qii′
STIQPRASGVQGFDISSYQGTVNFAGAYGAGARFVMIKATEGTTYIDSTFSSHYDGATSAGLIRGAYHFAHPDSSSGATQAEYFLAHGGGWTNDGITLPGMLDIEYNPSGSTCYGLSASAMVSWIKDFGETYNSKTGRYPMIYSTADWWSTCTGDSTSFSSDYPLVLAQYASSISTVPGGWPYQSFWQNADSYSYGGDSDLWNGSEDSLKTFAKGS

SEQ ID NO: 21 - protein, 218 aa, Aspergillus sp. nov XZ2609
LPTKLAARYSTVQGFDVSNYQPNVDFSAAKSAGAEFVIIKATEGTDYKDTYFNSHYTGATNAGLIRGGYHFARPDKSSGTAQAEYFLAHGGGWSKDGRTLPGMLDIEYNPYGATCYGLSHSAMVSWVNEFLNEYHSKTGVYPLLYTTADWWNQCTGNAHGFGNKSPLVLASYSSESPRTVPGDWQTWTIWQNADKYKYGGDSDIFNGDLTQLKKIVEG

SEQ ID NO: 22 - protein, 204 aa, Paecilomyces sp. XZ2658
TVAGFDISNYQPSVNFAKAYADGARFATEGTTYIDPSFSSHYTGATNAGLIRGGYHFAHPGSSTGAAQATYFLAHGGGWSKDGITLPGMIDLEYNPSGATCYGLSTSAMVSWISDFVETYHSKTGVYPLIYTSTSWWNQCTGSSTAFASKCPLVVARYASSVGTLPAGWGYQTIWQNSDSSPWGGDNDIFNGSLDQLKRIANAS

SEQ ID NO: 23 - protein, 203 aa, Paecilomyces sp. XZ2658
AVQGHDVSHWQGNINWGAVKAAGVKFTYIKATESTNYIDPSFNANYVGATNTGLIRGAYHFARPGDSSGAAQANYFVSHGGGWSADGRTLPGALDLEAGCSGLSQSAMTAWIRDFSNTYHARTGRFPVIYTTTSWWKTCTGNASGFQNDHPLWIARWGPSPGELPAGYGFHTFWQYADKGPLPGDQDNFNGDEAGLARLARGS

SEQ ID NO: 24 - protein, 208 aa, Pycnidiophora cf dispera
AVSGMDISHYQGTNYNFAGAYSSGARFVIIKATEGTTYTDPQFSANYIGATNAGFIRGAYHFARPAASTGAVQASYFVSHGGGWSSDGITLPGMLDMEYGSTSTCHGLSVSAMNTWIASFVNQYKSLTGAYPMIYTTADWWKTCTGDSTAWNTKCPLVLARYSSSVGTIPGGWPYHTIWQYSDSYAYGGDSDTFNGDLAGLKRLAKGS

SEQ ID NO: 25 - protein, 210 aa, Thermomucor indicae-seudaticae
YQTGLDVSALTSTSSFSCAKNLGYDHVIARCYMEAYGNNPGGKVDPNCYSNYKNAKAAGFTSVDIYMFPCTGRSTCKSPATQVQEIVDYVGAHKMIVGTLWLDVEVDSAANNWPSTSEARSTLRAFKTALDKSGWKWGVYSSKSQWTRITGSASWVLDPSVPLWYSHYDDTLSFSDYPSHAFGGWSKPTIKQYTGDASFCSASWDKNYYG

SEQ ID NO: 26 - protein, 207 aa, Isaria farinosa
LTHAVDSSSEVSVDIYKKALGQGFTRAIFRGYQEACSLGGRVDPTFVPSYKNAVAAGYKDFDAYFFPCTGTTNKCKPYATQLAELLDTISSQKLAIRRIWLDIETDQVCSPFDYGAQGNIAEAKKLVAAFRAAKHDWGIYTSPTQWETIFGSKTFVLANDVPLWFAKFDNVETLDLKTPFGGWTKADAKQYTDQSASKKFDLNVFSA

SEQ ID NO: 27 - protein, 207 aa, Lecanicillium sp. WMM742
SVQGFDISHYQSSVNFGAAYADGARFVIIKATEGTTYRDPKFSEHYGGATKAGFIRGGYHFAQPASSSGAAQANFFLAHGGGWSGDGITLPGMLDLEYGPNGNTCYGLGPASMRSWISDFVETYHAKTGRYPLIYTSTSWWKTCTGNTSLFADKCPLVVARYNSQVGELPAGWGFYTFWQFNDHYKHGGDSDVFNGAYSQLQKIATG

SEQ ID NO: 28 - protein, 208 aa, Zopfiella sp. t180-6
AVQGFDVSHWQSSVNFAAAYNSGARFVIIKATESNNYIDPKFNTYYPAATSAGLIRGGYHFAHPGETTGAVQADYFIAHGGGWSSDGITLPGMLDLENASGYPACWGLSQSAMVSWIKAFSDRYKARTGVYPMLYTNPSWWTSCTGNSNAFVNTNPLVLARYASSPGTIPGGWPYQTIWQNSDSYTYGGDSDIFNGDLAGLKRLAKGS

SEQ ID NO: 29 - protein, 217 aa, Malbranchea flava
GPKEFESRASGVQGFDISGWQSNVNFAGAYNSGARFVMIKASEGTTFKDRQFSNHYIGATKAGFIRGGYHFALPDVSSATAQVNHFLASGGGWSRDGITLPGMLDIESNPYGAQCYGLDAGRMVAWIREFVDAYKRATGRYPLIYTSPSWWQTCTGNSNAFIDKCPLVLARWASSPGTPPGGWPFHSFWQYADSYQFGGDAQVFNGDEAGLKRMALG

SEQ ID NO: 30 - protein, 208 aa, Hypholoma polytrichi
LVYGVDSSSLVPVATYQKALGEGFTKAVIRGYEEACGVGGEVDPNFVPSYKNARAAGYTDIDMYWFPCNGSTHSCKSYAAQLAAIAAAFSANAMKIGTIWIDIEKDAAICNNWDYGTAGNLAQAKALIAAAKASGFNFGIYSSPGEWSTIFGSTSVVVDNSAPLWFATYNNVQTLTLGTPFGGWSTAVGHQYTDVSASGLFDLNVFAH

SEQ ID NO: 31 - protein, 201 aa, Aspergillus deflectus
AVQGFDISHYQSSVDYAGAYSSGARFVMIKATEGTTYTDPAFSTHYTGATNAGLIRGGYHFARPGSSSGAAQAEYFLAHGGGWTGDGITLPGMLDLEAGCSGLSASAMVSWIQDFGETYKASTGRYPMIYTTTSWWSSCTGNNGGFGDYPLVLARWASSPGELPNGWSVHSFWQNADTYEYGGDSEIWNGSQENLVKFASQ

SEQ ID NO: 32 - protein, 202 aa, Ascobolus stictoideus
AVPGFDISHYQSTVNFADAYSSGARFVIIKATEGTTYKDPKFSSHYTGATNAGLIRGGYHFARPASSTGAVQAQYFVSNGGGWSGDGLTLPGMLDLEGDCAGLSQAGMVSWITSFVNKYKALTTRYPMIYTTNSWWNTCTGNSQAFSANCPLVIARYNSVVGTIPGGWPYYTIWQFNDAYSYGGDSDTFNGAYSQLVKLATG

SEQ ID NO: 33 - protein, 207 aa, Coniochaeta sp
TVQGFDISHYQPTVNYAGAYNAGARFVIIKATEGTTYTDPSFSTHYNGATKAGLIRGGYHFAHPGVTTGAAEANFFLAHGGGWSGDGITLPGMLDLESEGSNPQCWGLSTSGMVAWIKSFSDRYHTVTGRYPMLYTNPSWWSTCTGNSNAFVNTNPLVLARYASAPGTIPGGWPYQTIWQNSDSYSYGGDSDIFNGNLASLQKLATG

SEQ ID NO: 34 - protein, 202 aa, Daldinia fissa
AVPGFDISHYQSSVDFGAAYSSGARFVIIKATEGTTYQDPKFSSHYAGATNAGLIRGGYHFARPASSSGAAQATFFLAHGGGWSGDGITLPGMLDLEGDCAGLSTSAMVSWIRDFSDTYHGKTGRYPLLYTNPSWWSSCTGGSSAFVNTNPLVLARYASSPGALPGGWPYYTIWQFNDAYKYGGDSDTFNGDLTQLKKLASG

SEQ ID NO: 35 - protein, 201 aa, Rosellinia sp
AVQGFDISHYQSSVNFAGAYSAGARFVIIKATEGTSYIDPKFSSHYIGATNAGLIRGGYHFAHLGSSSGAAQANYFLAHGGGWSGDGITLPGMLDLEGDCVLSASGAVAWIKDFSDTYHSKTGVYPLLYTNPSWWSSCTGNSNAFVNTNPLVLARYSSSAGTPPGGWPYYTIWQYNDAYAYGGDSDVFNGDMAGLLRLAKG

SEQ ID NO: 36 - protein, 202 aa, Ascobolus sp. ZY179
AVPGFDISHWQSSVNFASAYSSGARFVIIKATEGTTYKDPKFSSHYTGATKAGFIRGGYHFARPASSTGAAQAQFFASNGGGWSGDGITLPGMLDLEGDCAGLSQSGMVSWISSFVNKYRSLTGRYPMIYTTNSWWVTCTGNSKAFSSNCPLVIARYNSVVGTIPGGWPYYTIWQYNDAYKYGGDSDTFNGAYSQLVKLATG

SEQ ID NO: 37 - protein, 206 aa, Curreya sp. XZ2623
TVPGFDISHYQGTVNFAGAYSSGARFVIIKATEGTTYTDPNFSNNYVGATNAKFIRGAYHFARPDGGSGSTQAQFFHSHGGGWSGDGITLPGMLDIEYGPTSTCYGLSTSAMVTWITDFVNEYHALTGRYPLIYTTNDWWNTCTGNTNKFSTTCPLVLARYSSSVGTIPGGWPFQTIWQFNDNYAYGGDSDTFNGDLAGLKKLATG

SEQ ID NO: 38 - protein, 202 aa, Coniothyrium sp
AVQGFDISHYQASVNFAAAYSGGLRFVYIKATEGTTYQDPAFSSHYSGATSAGFIRGGYHFARPASSTGAAQASYFVAHGGGWSNDGITLPGMLDLEGDCAGLSTASMVSWISSFSNQYHSLTGRWPVIYTTNSWWTTCTGNSAAFNANSPLMLARWGSTAGTIPGGWPYYTIWQYKDSNTYGGDSDVFNGDATQLKKLATG

SEQ ID NO: 39 - protein, 202 aa, Hypoxylon sp
AVPGFDISHYQSNVDFGAAYSSGARFVIIKATEGTTYQDPSFSTHYTGATKAGLIRGSYHFARPGSSSGAAQATYFLAHGGGWSGDGITLPGMLDLEGDCAGLSTNAMVAWIRDFSDTYHGRTGRYPLLYTNPSWWSGCAGGSAAFVGTNPLVLARYAGSPGALPGGWPYYTIWQFDDAYKYGGDSDTFNGDLTQLKKLASG

SEQ ID NO: 40 - protein, 202 aa, Xylariaceae sp. 1653h
AVPGFDISHYQSSVDFAAAYSAGARFVIIKATEGTTYIDPSFSSHYTGATNAGLIRGGYHFAHPGSSSGATQANYFLAHGGGWSGDGITLPGMLDLEGDCAGLSTSAMVSWIKDFSNAYHSKTGRYPLLYTNPSWWSSCTGSSSAFVNTNPLVLARYSSSAGTPPGGWPYYTIWQFNDAYKYGGDSDTFNGEYASLQKLATG

SEQ ID NO: 41 - protein, 202 aa, Hypoxylon sp
AVPGFDISHYQSSVDFAAAYNSGARFVIIKATEGTTYIDPSFSSHYTGATKAGFIRGGYHFAHPGSSSGAAQANYFLAHGGGWSGDGITFPGMLDLEGDCAGLSTSAMVSWIKDFSDTYHSKTGRYPLLYTNPSWWSSCTGDSSAFVNTNPLVLARYSSSAGTPPGGWPYYTIWQFNDAYKYGGDSDTFNGNYASLQKLATG

SEQ ID NO: 42 - protein, 206 aa, Yunnania penicillata
DVDGFDISHYQETVDYAGAYGAGARFVIIKATEGTNYIDSSFNTHYAGATDAGLIRGGYHFAHPGETTGAEQADYFIAHGGNWSNDGITLPGMLDLEGEGSTTCWDLSAADMVAWIKAFSDRYQEVTSRYPLLYTNPSWWSECTGNSDAFVDTNPLVLARYASSPGEIPGGWPAQTIWQNSDSYSFGGDSDIFNGDEAGLKKLASG

SEQ ID NO: 43 - protein, 207 aa, Engyodontium album
RVQGFDISHYQPSVDFNAAYADGARFVIIKATEGTTYKDPKFSQHYIGATNAGFIRGGYHFAQPASSSGAAQADYFLKNGGGWSSDGITLPGMLDMEYNPNGSACYGLSQASMRNWINDFVNTYHSRTGVYPLLYTTTSWWKTCTGNTAMFADKCPLVIARYNSVVGELPAGWSFWTIWQYNDHYKHGGDSDAFNGDYSQLQRIARG

SEQ ID NO: 44 - protein, 208 aa, Metapochonia bulbillosa
TVAGFDISNYQPTVDFKKAYADGARFVIIKATEGTTYTDPSFSSHYTGATQAGLIRGGYHFAHPGSGTGAAQANYFLAHGGGWSKDGITLPGMIDLEYNPSGATCYGLSASGMVSWISDFVETYHSKTGVYPLIYTSTSWWNQCTGSSTAFGNKCPLVVARYASSVGALPAGWGFQTIWQNSDKSPWGGDNDIFNGSLDQLKRIANAS

SEQ ID NO: 45 - protein, 215 aa, Hamigera paravellanea
APLEARAGSVQGFDISHYQAKVDFAAAYRSGARFVIIKATEGTTYTDPAFSSHYTSATNAGFIRGGYHFAHPDSSSGAAQATYFLAHGGGWSGDGITLPGMLDLEYNPSGATCYGLSDAAMVAWIQDFVDTYHARTGRYPMIYTTADWWNTCTGNSSKFSQTCPLVLARYASSVGTVPGGWGYQTIWQNSDSYAYGGDSDIFNGDETQLKKLASG

SEQ ID NO: 46 - protein, 217 aa, Metarhizium iadini
SPVELEQRAASVKGFDISGYQPNVDFNKAYADGARFVIIKATEGTTYIDKTFSKHYTGATKAKLIRGAYHFAHPGQNKASAEADFFVQHGGNWSKDAITLPGMVDLESEKGHPPCWGLSHSAMVAWISEFVAAYHKKTTRYPMLYTNPSWWSACTGNSKAFKDTCPLVLARYASSPGAIPGGWPAQTIWQNSDKSPWGGDSDMFNGDLAQLKKLATG

SEQ ID NO: 47 - protein, 214 aa, Thermoascus aurantiacus
ELDKRARGVQGFDISHYQPNVDFKGAYNSGARFVIIKATEGTTYKDPAFSKHYIGATEAGLIRGGYHFAHPDKSSGAAQANFFLAHGGGWSGDGITLPGMVDLEYNPSGDACYGLSDSQMVSWIRDFVNTYHAHTGRYPMIYTTADWWKRCTGDSHAFSTTCPLVLARYNSSPGTVPGGWPYHTIWQNSDKYRFGGDSDIFNGDLAGLKRLAKG

SEQ ID NO: 48 - protein, 208 aa, Clonostachys rossmaniae
AVPGFDISGWQKSTDFAKSYANGDRFVYIKATEGTTFKNPLFSKQYTGATNARLIRGAYHFAQPASSSGASQARFFVANGGGWSNDGITLPGAVDMEYNPSGATCYGLSKTAMVNWIEDFVSTYQALTGRWPVVYTTLDWWTQCTGNSAKFGDRCPLWVARYASAVGQIPAGWSFHTIWQYNAKYPEGGDSDIFNGDETRLKALASGA

SEQ ID NO: 49 - protein, 203 aa, Simplicillium obclavatum
APKGIDVSHWQGSINWGAVKANGIEWAYIKATESTNYKDPNFNANYVGATNAGLIRGAYHFARPGDSSGAAQANYFASNGGGWSADGITLPGAVDLEAGCSGLSQSAMTAWIKDFSNTYHARTGRYPAIYTTTSWWKQCTGNASGFQNNNPLWIARWASSVGELPAGYSYHTFWQYADHGPNPGDQDVFNGDSAGLKRMAKGS

SEQ ID NO: 50 - protein, 216 aa, Aspergillus inflatus
APLEARANTVQGFDISSFQPNVDFAAAYKAGARFVMMKATQNTNYVDKTFNAHYEGATKAGLIRGGYHFAIPNGPSGAAQAEYFLAHGGGWSDDGKTLPGMIDLEYNPYGQTCYDLSAAKMVDWIKDFSNTYHAKTKRYPMIYTTANWWKECTGDSKEFSQTNPLVLARYSSSAGTVPGGWPAYSFWQNADKYKFGGDSDIWNGSEDNLKKFAKGA

SEQ ID NO: 51 - protein, 207 aa, Paracremonium inflatum
KVLGFDISHYQATVDFNAAKDAGARFVIIKATEGTTYKDPAFSKHYTGATKAGLIRGGYHFAQPASSSGAAQATFFLAHGGGWSSDGITLPGMLDLEYNPSGSTCYGLSQSSMVQWISDFIDTYHSKTGRYPLIYTSTSWWKTCTGNSSKFAANCPLVVARYSSSVGELPAGWTYYTIWQNSDSYKYGGDSDIFNGDESQLQKLAKG

SEQ ID NO: 52 - protein, 208 aa, Westerdykella sp
AVSGMDISHYQGTNYNFAGAYSSGARFVIIKATEGTTYTDPQFSANYIAATNAGFIRGGYHFARPADSTGAAQAKYFVSHGGGWSSDGITLPGMLDLEYGSSSACHGLSVSAMNTWIASFINQYRSLTGAYPMIYTTADWWKTCTGDSQAWNTKCPLVLARYSSSVGTIPGGWPYQTIWQFNDSYKYGGDSDTFNGDLAGLKRLAKGS

SEQ ID NO: 53 - protein, 207 aa, Stropharia semiglobata
LTYAVDSSTLVSVATYTKAKSQGFTKAIIRGYQEACGSGGAVDPNFVQTYKNARAAGYTDIDMYWFPCNGSTHNCKSYATQIAAIAATFSANSMKIGRIWIDIEKDAAVCNNWNYGTAGNLSQAKALISAIKASGFVYGIYSSPGEWGNIFGSTSVVVDNSAPLWFATWNNVQTLTMGTKFGGWTSAMGHQYTDVSASGQFDLSVFA

SEQ ID NO: 54 - protein, 208 aa, Gelasinospora cratophora
TVQGFDISHYQSSVNFAGAYSSGARFVIIKATEGTTYIDSSFSSHYTGATSAGLIRGGYHFAHPDSSTGAAQADYFLAHGGGWSADGITLPGMIDLESVSGKATCFGLSTSAMVSWIKSFSDRYYAKTGRYPMIYTNYSWWNQCTGNSASFAATNPLVLARWSSTVGTLPGGWSVQTIWQNADTYTYGGDSDVFNGSLDRLKALAKGS

SEQ ID NO: 55 - protein, 207 aa, Flammulina velutipes
RLNGIDVSGYQPNVNWATVKANGVSFAYIKATEGTTYTNPSFSSQYTGATKAGLIRGSYHFAHPSSSTGAAQARYFVAHGGGWSGDGITLPGALDIEYNPSGATCYGLSTSSMVNWIADFSNTYHSLTGRYPVIYTTADWWRTCTGNSASFANNSPLWIARYASTIGTLPAGWSYATFWQYADSGSNPGDQDYFNGDAAGLKRLATS

SEQ ID NO: 56 - protein, 207 aa, Deconica coprophila
LVHAVDSSSLVSTATFSKAKSEGFTKAVIRGYQEACGSGGRVDPNFVQTYKNARAAGITNIDTYWYPCNGSGNSCKSYAKQIAGISATFNAHSMKIGRIWIDIEKDSICNNWNYGTSGNRDHAKKLITAIKNSGFKYGIYSSPGEWSTIFGSESFDLDSGAPLWFATWNNVQTLTLGTHFGGWTSAHGHQYTDKSASGQFDLNVFSS

SEQ ID NO: 57 - protein, 208 aa, Rhizomucor pusillus
YETGVDVSALTSTSAWSCAKKLGYDHAIVRCYIEAYGGNPGGKIDSNCFQNYKNAKAGGFTSVDIYMFPCTGRSTCKSPAAQVKEVVDYVGSNKMTVGRLWLDVEIDPSANNWPSASSARSTLKSFKSALDSTGWKYGIYSSASQWSQITGSSSWELDSSLPLWYAHYDASLSFSDFSPFGGWTKPTIKQYAGSVSFCSAGWDKNYYG

SEQ ID NO: 58 - protein, 207 aa, Stropharia semiglobata
LVYGVDSSTLVSTATYSKAKSEGFTKAIIRGYQEACGSGGRVDPNFVATYKNARAAGITDIDMYWFPCNGSGNSCKSYAKQLSEIANVFSANSMKIGTIWIDFEKDSGCNNWNYGTTGNLNHAKALISAIKATGFKFGIYSSPGEWGTLFGSTGVVLDSSAPLWFATWNNVKTLTLGTHFGGWTKAVGHQYTDVSASGQFDLNVFAN

SEQ ID NO: 59 - protein, 207 aa, Stropharia semiglobata
LVYGVDSSTLVSTATYKKAKSEGFTKAIIRGYQEACGSGGRVDPNFVATYKNARAAGITDIDMYWFPCNGSGNSCKSYAKQLSEIANVFSANSMKIGTIWIDFEKDSGCNNWNYGTTGNLNHAKALISAIKATGFKFGIYSSPGEWGTLFGSTGVVLDSSAPLWFATWNNVKTLTLGTHFGGWTTAAGHQYTDVSSSGQFDLNVFAN

SEQ ID NO: 60 - protein, 207 aa, Myceliophthora fergusii
AVQGFDISHWQSSVDFKAAYNSGARFVIIKATEGTSFIDPKFSSHYTGATNAGFIRGAYHFAHPGQSSGEAQADYFLAHGGGWTPDGITLPGMLDLEAYNAGECWGLSQSAMVAWIKAFSDRYHARTGVYPMLYTNLSWWKTCTGNSKAFVNTNPLVLARWASSPGEIPGGWPWQTIWQNSDSYRYGGDSDIFNGDMNQLRRLATAA

SEQ ID NO: 61 - protein, 204 aa, Mortierella alpina
ALPKGIDVSHWQGDVNWNSVKAAGIEFVYIKATESINYIDSKFDANYVGATNAGLIRGGYHFARPAASSGAVQANYFLANGGGWSSDGITLPGALDLEAGCSGLSQAAMTAWVRDFSDTYHARTGRYPVIYTTTSWWKQCTGNASGFQNNNPLWIARWASSAGELPAGYAFHTFWQYADKGPNPGDQDYFNGDSAGLRRFAKGS

SEQ ID NO: 62 - protein, 216 aa, Penicillium atrovenetum
TPLESRASGVQGFDISSYQGTVDFAGAYAAGARFVMIKATEGTTYTDKTFSSHYEGASSAGLIRGGYHFAHPDSSSGAKQAEYFLAHGGGWSNDGKTLPGMLDIEYNPSGATCYGISKSAMVAWVKDFGETYKGKTGRYPMIYTTADWWNTCTGGSTAFSKDYPLVLARYSSSVGTIPGGWPYQSFWQNSDKYTYGGDSDLWNGSEASLKTFAKGA

SEQ ID NO: 63 - protein, 245 aa, Trichophaea saccata
YPVKTDLHCRSSPSTSASIVRTYSSGTEVQIQCQTTGTSVQGSNVWDKTQHGCYVADYYVKTGHSGIFTTKCGSSSGGGSCKPPPINAATVALIKEFEGFVPKPAPDPIGLPTVGYGHLCKTKGCKEVPYSFPLTQETATKLLQSDIKTFTSCVSNYVKDSVKLNDNQYGALASWAFNVGCGNVQTSSLIKRLNAGENPNTVAAQELPKWKYAGGKVMPGLVRRRNAEVALFKKPSSVQAHPPKC

SEQ ID NO: 64 - protein, 249 aa, Chaetomium thermophilum
PASAYAITGDNVNCRSGPGTSYAVKKVYKKGTDVKISCQTTGTNINGNNLWDKTSDGCYVSDYYVKTGSNGYVTSKCSSSGGSTCAAPKSNQATVDLIAEFEGFRANIYTDAAGYATVGYGHKCQKAKCAEVKYKIPLSKADGKKLLADDMRSFEVCITNMLNSKAKLNYNQFGALVSWSFNVGCGAAKSSTLIKRLNNGENVNKVLSEELPKWNKAGGKVLQGLVRRRAAEVALAKKSGSSQALPVKC

SEQ ID NO: 65 - protein, 248 aa, Trichoderma harzianum
YPITGDVVNCRTGPGTSYAIKKSYKKNQDISISCQTAGTSVNGNSIWDKTADGCYVADYYVKTGSSGYVTKKCTASSGGGSSSSYCKTINSAGVDLIAKWEGFVASPKPDPIGLPTVGYGHLCQQKNCREVKYKFPLTKTTAKELLLDDLPKYTKCLADYLNDKPKLNANQWAALTSWVFNVGCGNAKTSTLVKRLNNGEAANTVAAEELPKWRMAGGKVLPGLEARRKDEVKLFKTASSKQAYPKCQ

SEQ ID NO: 66 - protein, 245 aa, Trichophaea minuta
YPAKVDLRCRSSPSTSASVVRTYSKGSEIQISCQTTGTSVEGSNVWDKTQHGCYVADYYVKTGHSGIFTTKCGSSSGGGSCKPPPINAATVALIKEFEGFVAKPAPDPIGLPTVGYGHLCKTKGCKEVPYSFPLTQTTATKLLQSDIKTFTSCVSNYVKDSVKLNDNQFGALSSWAFNVGCGNIQTSSLIKRLNAGENPNTVAAQELPKWKYAGGKVLPGLVRRRKAEVALFKKPSSVQAHPPKC

SEQ ID NO: 67 - protein, 249 aa, Chaetomium sp. ZY287
YKISGSSVNCRSGPGTNYPVKKTYANGDEVTISCQTTGTNVEGNNIWDKTQHGCYVADKYVKTGKDGFVTKKCGSSGGGGGGKTCKAPKSNAATVDLIASFEGFRANIYTDATGHPTVGYGHMCTKSRCAEVKYKIPLSKADGKKLLADDMAKFEKCIKEMLNSKAKLNLNQYGALVSWSFNVGCGAAKGSQLVSRLNKGENPNTVLSNELPKWVHGNGKVLPGLVRRRNAEIALAKKSGSGAALPVKC

SEQ ID NO: 68 - protein, 245 aa, Mortierella sp. ZY002
YPITGADALHCRSGPGTSYPIQKTLRPPQDIKIQCQEPGTVVNGVSLWDKTQFGCYVSDYYVKTGTGNYVAPRCNSGGSSSACTGLNDAGINLIKEFEGFVPRPAPDPIGLPTVGYGHLCQTKGCGEVKYSFPLTTATATALLKDDLPKYTSCLAKALNGKPKLNKNQWAALASWTFNVGCGNMKSSSLITRLNAGQNPNTVATEELPKWKLAGGKVLPGLVRRRAAEVKLFKTANSSQGYPKCA

SEQ ID NO: 69 - protein, 247 aa, Metarhizium sp. XZ2431
YPVSADSLNCRAEPNTSSAIKTTYKKGEDVKISCQTEGPSINGNTIWDKTQDGCYVADYYVKTGSSGYVTGKCGGSSPPSGSGFCKTVNKAGLDLITKWEGFVSSPRGDPIGLPTVGYGHLCQKKGCAEVKYKFPLTKATALQLLNDDLPKYTGCLGKLLNSKVKLNDNQWAALTSWVFNVGCGNAQSSSLVRRLNNGENPNTVAPSELPKWKMAGGKVLEGLVKRRADEVRLFKVSSSKGAFPKCQ

SEQ ID NO: 70 - protein, 250 aa, Geomyces auratus
AFPITGSTVNCRTGPGTSHGVKTSYKKGHEVTVSCQTGGTSVNGNSIWDKTSDGCYVADYYVKTGSSGYVKPKCGSSSGGGGGSSCGAPKSNAATVNLIAEFEGFVSHVYTDATGHPTVGYGHLCSNSKCSGIGYSIPISKANAKKLLAKDMAIAEKCITAMINKSRTLNLNQYGALVSWAFNEGCGAAKSSTLIKRINNGEKPSTVIPQELPKWVYGGSSVLPGLVRRRNAEIALAKKATSSKALPAHC

SEQ ID NO: 71 - protein, 240 aa, Ilyonectria rufa
YKITGDNVNCRSGPGTSYSVKRSFKKGTDVTLSCQTTGENVLGTSIWDKTSYGCYVSDYYVKTGSSGFVVKKCGTCGAPKSNAATVNLISDFEGFRANIYKDAAGYPTVGYGHLCSNSRCTDVPYSIPLSKANGKNLLATDMTKFEKCITAMVSSSVTLNKNQYGALVSWAFNMGCGATKTSTLIKRLNQGQNVNTVLSTELPKWVYAGGKKLNGLVRRRNAEIALAKKKTTEKALPNKC

SEQ ID NO: 72 - protein, 188 aa, Nocardiopsis sp. NRRL 18262
ADIIGGLAYTMGGRCSVGFAATNAAGQPGFVTAGHCGRVGTQVTIGNGRGVFEQSVFPGNDAAFVRGTSNFTLTNLVSRYNTGGYAAVAGHNQAPIGSSVCRSGSTTGWHCGTIQARGQSVSYPEGTVTNMTRTTVCAEPGDSGGSYISGTQAQGVTSGGSGNCRTGGTTFYQEVTPMVNSWGVRLRT

SEQ ID NO: 73 - protein, 17 aa, Nocardiopsis alba
ADIIGGLAYTMGGRCSV

SEQ ID NO: 74 - protein, 314 aa, Bacillus horneckiae
EVTATPSTQTPWGIKSIYNDQSITKTTGGSGIKVAVLDTGVHTGHIDLAGSSEQCKDFTQSNPLVNGSCTDRQGHGTHVAGTVLAHGGSDGQGVYGVAPQAKLWAYKVLGDNGSGYSDDIAAAIRHVADEASRTGSKVVINMSLGSSGKDSLIASAVDYAYGKGVLIVAAAGNSGSGSNTIGYPAALVNAVAVAALENVQQNGTYRVANFSSRGNPATAGDFRIQERDVEVSAPGASVESTWYNGGYNTISGTSMATPHVAGLAAKIWSSNSSLSHSQLRTELQNRAKVYDIKGGIGAGTGDDYASGFGYPRVK

SEQ ID NO: 75 - protein, 311 aa, Bacillus sp. [sequence omitted]

SEQ ID NO: 76 - protein, 311 aa, Artificial Sequence
  Protein Engineered variant
AVPSTQTPWGIKSIYNDQSITKTTGGKGIKVAVLDTGVYTSHLDLAGSAEQCKDFTQSNPLVDGSCTDRQGHGTHVAGTVLAHGGSNGQGVYGVAPQAKLWAYKVLGDKGEGYSDDIAAAIRHVADEASRTGSKVVINMSLGSSAKDSLIASAVDYAYGKGVLIVAAAGNEGPKPNTIGYPAGFVNAVAVAALENVQEKGTYRVADFSSRGNPATAGDYIIQERDIEVSAPGASVESTWYTGGYNTISGTSMATPHVAGLAAKIWSANTSLSHSQLRTELQNRAKVYDIKGGIGAGPGDDYASGFGYPRVK